(19) United States
(12) Patent Application Publication
(10) Pub. No.: US 2025/0109115 A1
(43) Pub. Date: Apr. 3, 2025

(54) METHOD FOR PREPARING A CYCLIC ANHYDRIDE FROM AN UNSATURATED CARBOXYLIC ACID

(71) Applicants: Commissariat a l'Energie Atomique et aux Energies Alternatives, Paris (FR); Centre National de la Recherche Scientifique, Paris (FR)

(72) Inventors: Marie-Hélène PIETRARU, Gif-sur-Yvette (FR); Louise PONSARD, Gif-sur-Yvette (FR); Nicolas LENTZ, Gif-sur-Yvette (FR); Emmanuel NICOLAS, Gif-sur-Yvette (FR); Thibault CANTAT, Gif-sur-Yvette (FR)

(73) Assignees: Commissariat a l'Energie Atomique et aux Energies Alternatives, Paris (FR); Centre National de la Recherche Scientifique, Paris (FR)

(21) Appl. No.: 18/708,444

(22) PCT Filed: Nov. 8, 2022
(86) PCT No.: PCT/EP2022/081174

§ 371 Date: May 8, 2024

(30) Foreign Application Priority Data
Nov. 12, 2021 (FR) FR2111996

(51) Int. Cl. C07D 307/60; B01J 31/04; B01J 31/18; B01J 31/20; B01J 31/24

(52) U.S. Cl. CPC C07D 307/60; B01J 31/04; B01J 31/181; B01J 31/20; B01J 31/24; B01J 2231/324; B01J 2531/004; B01J 2531/824; B01J 2531/845

(57) ABSTRACT

This invention relates to a method for preparing a cyclic anhydride such as succinic anhydride and methyl succinic anhydride from an unsaturated carboxylic acid such as acrylic acid or crotonic acid. This method can also be used in the manufacture of food additives, plasticisers, polymers of interest, in particular polyurethanes and elastanes, resins, coatings and pharmaceutical products.

### TECHNICAL FIELD OF THE INVENTION

This invention relates to a method for preparing a cyclic anhydride such as succinic anhydride and methyl succinic anhydride from an unsaturated carboxylic acid such as acrylic acid or crotonic acid. This method can also be used in the manufacture of food additives, plasticisers, polymers of interest, in particular polyurethanes and elastanes, resins, coatings and pharmaceutical products.

### TECHNICAL BACKGROUND

The succinic anhydride is a molecule of industrial interest. The succinic anhydride is used as a monomer, for example, in the synthesis of aliphatic polyesters via a catalytic copolymerisation with an epoxide. The succinic anhydride can also be an intermediate in the synthesis of other molecules of interest: its hydration gives the corresponding diacid, succinic acid, and its dehydrogenation gives γ-butyrolactone(GBL) and then 1,4-butanediol (BDO), which can in turn be dehydrated to tetrahydrofuran (THF). These reactions are illustrated in FIG. 1.

Currently, the industrial method used for the synthesis of succinic anhydride is based on the hydrogenation of the maleic anhydride catalysed by metal complexes typical of hydrogenation reactions, such as Raney nickel or palladium, at temperatures ranging from 120 to 180° C., and at dihydrogen pressures ranging from 5 to 40 bar (Fumagalli, C. Succinic Acid and Succinic Anhydride. In Kirk-Othmer Encyclopedia of Chemical Technology; 2006 (doi: 10.1002/0471238961.1921030306211301.a01.pub2).

Other succinic anhydride synthesis methods are illustrated in FIG. 2.

The current routes of synthesising succinic anhydride have certain disadvantages. In the case of the synthesis route most commonly used in the industry, which is the hydrogenation of maleic anhydride, the latter is a petroleum-based compound, and the reaction requires a catalysis using precious metals, under high temperature conditions (above 120° C.), which increases the energy consumption required for the synthesis.

Yamamoto et al. (Yamamoto, T., Igarashi, K., Komiya, S., & Yamamoto, A., Journal of the American Chemical Society, 1980, 102(25), 7448-7456 (doi10.1021/ja00545a009)) described the carbonylation of acrylic acid leading, in several steps, to succinic anhydride as shown in FIG. 3. This reaction requires a stoichiometric quantity of nickel, and special conditions for the acrylic acid, in particular a temperature below room temperature (20-25° C.) to prevent its polymerisation. Apart from the polymerisation of acrylic acid, the other disadvantages of this synthesis route are the toxicity of the nickel used in stoichiometric quantities and the presence of multiple steps requiring intermediate purifications, resulting in low yields and high processing and separation costs.

There is therefore a real need for a method that allows the succinic anhydride to be synthesised in a sustainable way using reagents that can be bio-sourced and under relatively mild pressure and temperature conditions to reduce the energy consumption required to synthesise the succinic anhydride.

The succinic anhydride synthesis method must be easy to implement and of industrial interest. While the current industrial method requires the use of precious metals, a method offering a choice between precious and non-precious metals would be very advantageous.

It should also be applicable to the synthesis of succinic anhydride derivatives.

### SUMMARY OF THE INVENTION

The aim of the present invention is precisely to meet these needs by providing a method for preparing a cyclic anhydride of formula (I)

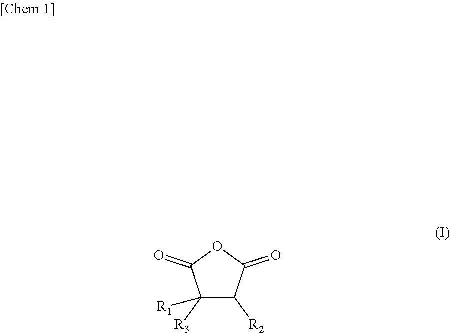

wherein
R₁, R₂ and R₃, which may be identical or different, represent a hydrogen atom, an alkyl radical containing 1 to 25 carbon atoms, a cycloalkyl radical containing 3 to 12 carbon atoms, an aryl radical containing 6 to 20 carbon atoms, a hydroxyl group, a nitrile group, an amine group, an amide group, a carbonyl group, a carboxylate group, a nitro (NO₂) group, an alkoxy group, an aryloxy group or a halogen atom, said alkyl, cycloalkyl and aryl radicals being optionally substituted; characterised in that an acid of formula (II)

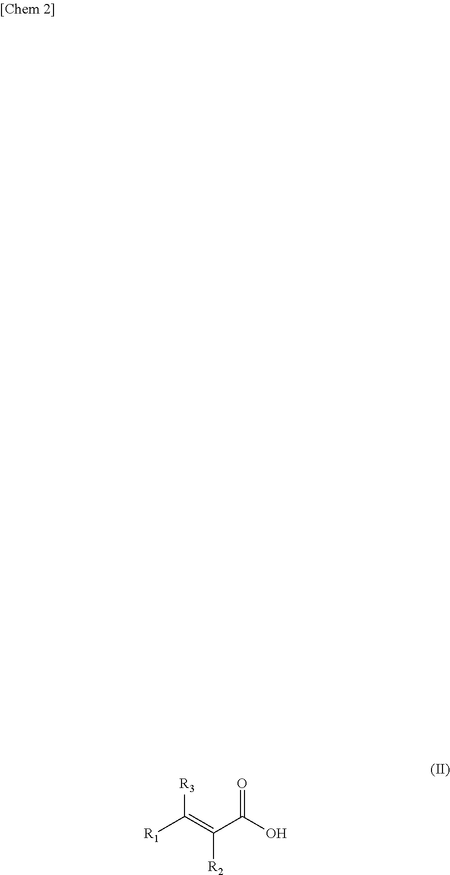

in which R₁, R₂ and R₃ are as defined above;
is reacted with carbon monoxide (CO) or a mixture of carbon monoxide and hydrogen (CO/H₂), in the presence of a metal catalyst of formula (III)

[Chem 3]

LₐM_{b}X_{c}(CO)_{d} (III)

wherein
L represents Na⁺, K⁺, Li⁺, Cs⁺, Mg²⁺, Ca²⁺, NH₄⁺, [((C₆H₅)₃P)₂N]⁺; a ligand chosen from:
the phosphines of formula PR₄R₅R₆, in which R₄, R₅ and R₆, which may be identical or different, represent a hydrogen atom, an alkyl radical containing 1 to 12 carbon atoms, an aryl radical containing from 6 to 20 carbon atoms, an alkoxy group, an aryloxy group or a ferrocenyl group, said alkyl, aryl and ferrocenyl radicals being optionally substituted;
the polyphosphines which mean a phosphine as defined above, in which at least one of the substituents R₄, R₅ and R₆ is substituted by one or several phosphinyl groups of formula —PR₁₁R₁₂, in which R₁₁ and R₁₂, which may be identical or different, represent an alkyl radical containing 1 to 12 carbon atoms, an aryl radical containing from 6 to 20 carbon atoms, an alkoxy group, an aryloxy group, said alkyl and aryl radicals being optionally substituted;
the amines of formula NR₇R₈R₉ in which R₇, R₈ and R₉, which may be identical or different, represent a hydrogen atom, an alkyl radical containing 1 to 12 carbon atoms or an aryl radical containing from 6 to 20 carbon atoms, said alkyl and aryl radicals being optionally substituted;
the amides of formula R₇CO—NR₈R₉ in which R₇, R₈ and R₉, which may be identical or different, represent a hydrogen atom, an alkyl radical containing 1 to 12 carbon atoms or an aryl radical containing from 6 to 20 carbon atoms, said alkyl and aryl radicals being optionally substituted;
the N-heterocyclic carbenes optionally derived from an imidazolium salt, chosen from the 1,3-bis(2,6-diisopropylphenyl)-1H-imidazol-3-ium salts (also referred to as Pr), 1,3-bis(2,6-diisopropylphenyl)-4,5-dihydro-1H-imidazol-3-ium (also referred to as s-IPr), 1,3-bis(2,4,6-trimethylphenyl)-1H-imidazol-3-ium (also referred to as IMes), 1,3-bis(2,4,6-trimethylphenyl)-4,5-dihydro-1H-imidazol-3-ium (also referred to as s-IMes), 4,5-dichloro-1,3-bis(2,6-diisopropylphenyl)-1H-imidazol-3-ium (also referred to as Cl₂—IPr), 1,3-di-tert-butyl-1H-imidazol-3-ium (also referred to as ItBu), and 1,3-di-tert-butyl-4,5-dihydro-1H-imidazol-3-ium (also referred to as s-ItBu), said salts being in the form of chloride or tetraphenylborate salts;
cyclopentadienyl and its derivatives selected from 1,2,3,4,5-pentakis(methyl)cyclopentadienyl, 1,2,3,4,5-pentakis(1-methylethyl)cyclopentadienyl and 1,2,3,4,5-pentakis(1-dimethylethyl)cyclopentadienyl;
the nitriles of formula NCR₁₀ in which R₁₀ represents an alkyl radical containing 1 to 12 carbon atoms or an aryl radical containing 6 to 20 carbon atoms, said alkyl and aryl radicals being optionally substituted;
the pyridine and its derivatives of formula (IV)

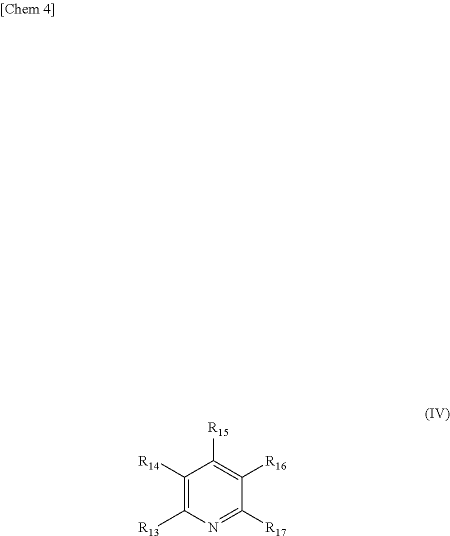

in which R₁₃, R₁₄, R₁₅, R₁₆ and R₁₇, which may be identical or different, represent a hydrogen atom, an alkyl radical containing 1 to 12 carbon atoms or an aryl radical containing 6 to 20 carbon atoms, said alkyl and aryl radicals being optionally substituted;
the bipyridines of formula (V)

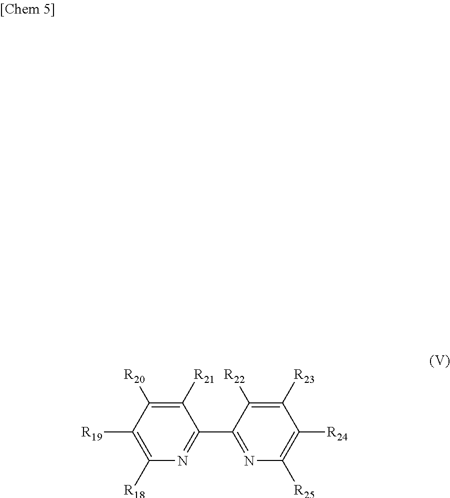

in which R₁₃, R₁₉, R₂₀, R₂₁, R₂₂, R₂₃, R₂₄ and R₂₅, which may be identical or different, represent a hydrogen atom, an alkyl radical containing 1 to 12 carbon atoms or an aryl radical containing 6 to 20 carbon atoms, said alkyl and aryl radicals being optionally substituted;
M represents Co, Fe, Mn, Rh, Ru, Pd; M representing in particular Co or Pd;
X represents a halide selected from F⁻, Cl⁻, Br⁻, I⁻; a trifluoromethylsulphonate (or triflate or CF₃SO₃⁻); an acetylacetonate (or acac or C₅H₇O₂⁻); an acetate (or CH₃COO⁻ or AcO⁻); a trifluoroacetate (or CF₃COO⁻ or TFAcO⁻);
a=0 to 12;
b=0 to 3;
c=0 to 12;
d=0 to 12;
in one or a mixture of at least two solvents chosen from:
the aromatic hydrocarbons chosen from benzene and toluene,
the nitriles chosen from acetonitrile, propionitrile and benzonitrile,
the linear or cyclic ethers chosen from diethyl ether, methyl vinyl ether, 1,1-diethoxyethane, dimethoxyethane, THF, methyltetrahydrofuran, tetrahydropyran and dioxane,
the esters such as ethyl acetate,
the carbonate esters such as propylene carbonate,
the ketones chosen from acetone and butanone,
the alkyl halides chosen from chloroform and methylene chloride,
the aryl halides chosen from chlorobenzene and dichlorobenzene;
at a temperature of 110° C. or less.

The conditions under which the method of the invention is implemented are particularly advantageous:
they allow to limit or even avoid the undesirable secondary reactions, namely oligomerisation or polymerisation of the acid of formula (II) and its hydrogenation (in the case of acrylic acid, to limit or even avoid its oligomerisation/polymerisation and its hydrogenation towards propionic acid);
the conditions used are mild, which allows to reduce the energy consumption required to synthesise the product;
an easy separation of the cyclic anhydride of formula (I) from the other species in the reaction medium is possible, in particular by choosing a solvent in which said cyclic anhydride has a very low solubility.

This method can also be used in the manufacture of food additives, plasticisers, polymers of interest, in particular polyurethanes and elastanes, resins, coatings and pharmaceutical products.

### BRIEF DESCRIPTION OF THE FIGURES

Further characteristics and advantages of the invention will become apparent from the following detailed description, for the understanding of which reference is made to the attached figures in which:

FIG. 1 shows the use of succinic anhydride as an intermediate in the synthesis of molecules of industrial interest.

FIG. 2 shows the current synthesis routes for succinic anhydride.

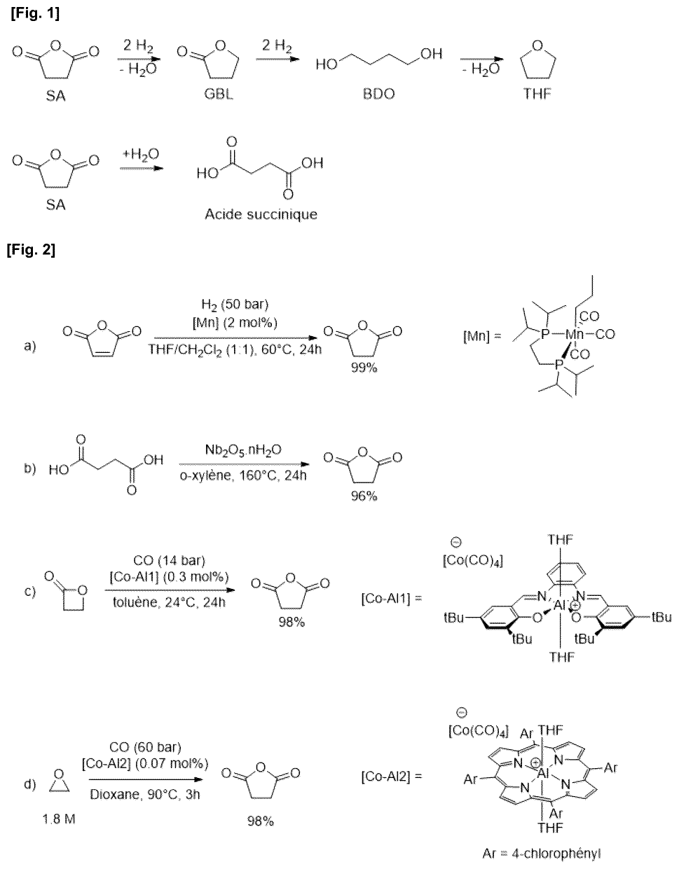

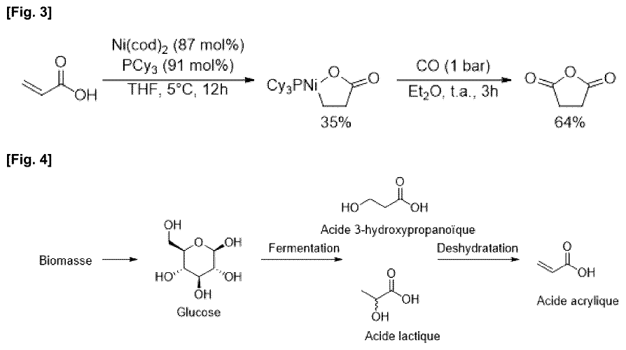

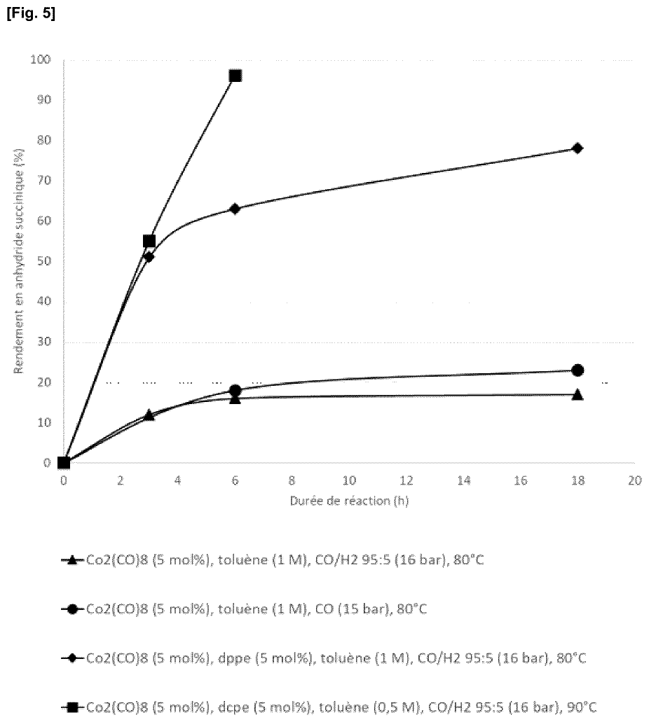

a) Hydrogenation of maleic anhydride under mild conditions, in the absence of precious metal (Weber, S.; Stöger, B.; Veiros, L. F.; Kirchner, K., ACS Catal. 2019, 9 (11), 9715-9720 (doi:10.1021/acscatal.9b03963)).

b) Dehydration of succinic acid (Rashed, M. N.; Siddiki, S. M. a. H.; Ali, M. A.; Moromi, S. K.; Touchy, A. S.; Kon, K.; Toyao, T.; Shimizu, K., Green Chem. 2017, 19 (14), 3238-3242 (doi:10.1039/C7GC00538E)).
c) Carbonylation of beta-lactone (Getzler, Y. D. Y. L.; Kundnani, V.; Lobkovsky, E. B.; Coates, G. W., J. Am. Chem. Soc. 2004, 126 (22), 6842-6843 (doi:10.1021/ja048946m)).
d) Double carbonylation of ethylene oxide (Rowley, J. M.; Lobkovsky E. B.; Coates, G. W., J. Am. Chem. Soc. 2007, 129 (16), 4948-4960 (doi:10.1021/ja066901a))

FIG. 3 shows the synthesis of succinic anhydride by carbonylation of acrylic acid by Yamamoto et al. (Yamamoto, T., Igarashi, K., Komiya, S., & Yamamoto, Journal of the American Chemical Society, 1980, 102(25), 7448-7456 (doi:10.1021/ja00545a009).

FIG. 4 shows the route for synthesising acrylic acid from biomass.

FIG. 5 shows the kinetics of succinic anhydride synthesis from acrylic acid under different conditions. Each point on the graph corresponds to a separate experiment. dppe=1,2-bis(diphenylphosphino)ethane; dcpe=1,2-bis(dicyclohexyl-phosphino)ethane.

### DETAILED DESCRIPTION OF THE INVENTION

The present invention relates to a method for preparing a cyclic anhydride of formula (I)

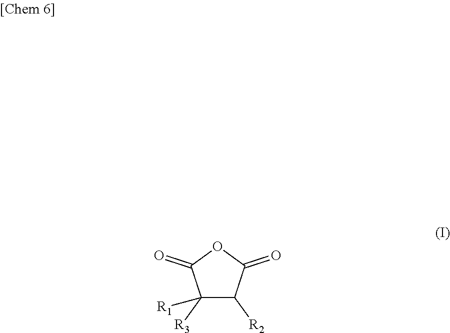

wherein
R₁, R₂ and R₃, which may be identical or different, represent a hydrogen atom, an alkyl radical containing 1 to 25 carbon atoms, a cycloalkyl radical containing 3 to 12 carbon atoms, an aryl radical containing 6 to 20 carbon atoms, a hydroxyl group, a nitrile group, an amine group, an amide group, a carbonyl group, a carboxylate group, a nitro (NO₂) group, an alkoxy group, an aryloxy group or a halogen atom, said alkyl, cycloalkyl and aryl radicals being optionally substituted; characterised in that an acid of formula (II)

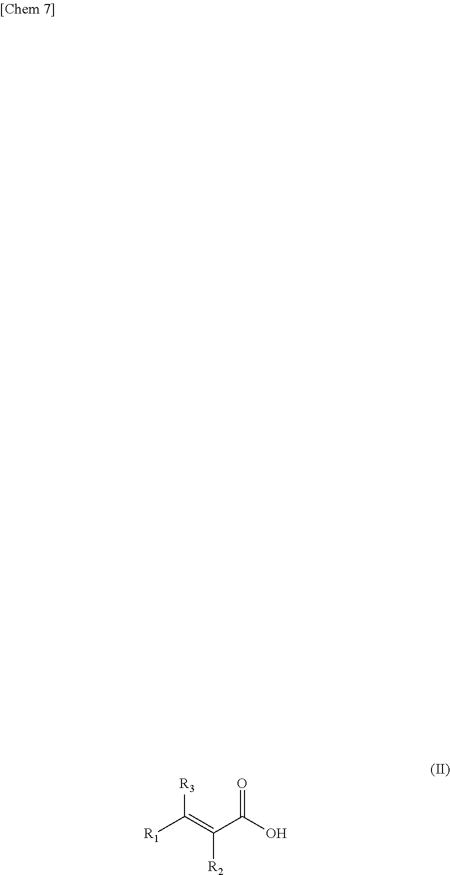

in which R₁, R₂ and R₃ are as defined above;
is reacted with carbon monoxide (CO) or a mixture of carbon monoxide and hydrogen (CO/H₂), in the presence of a metal catalyst of formula (III)

[Chem 8]

LₐM_{b}X_{c}(CO)_{d} (III)

wherein
L represents Na⁺, K⁺, Li⁺, Cs⁺, Mg²⁺, Ca²⁺, NH₄⁺, [((C₆H₅)₃P)₂N]⁺; a ligand chosen from:
the phosphines of formula PR₄R₅R₆, in which R₄, R₅ and R₆, which may be identical or different, represent a hydrogen atom, an alkyl radical containing 1 to 12 carbon atoms, an aryl radical containing from 6 to 20 carbon atoms, an alkoxy group, an aryloxy group or a ferrocenyl group, said alkyl, aryl and ferrocenyl radicals being optionally substituted;
the polyphosphines which mean a phosphine as defined above, in which at least one of the substituents R₄, R₅ and R₆ is substituted by one or several phosphinyl groups of formula —PR₁₁R₁₂, in which R₁₁ and R₁₂, which may be identical or different, represent an alkyl radical containing 1 to 12 carbon atoms, an aryl radical containing from 6 to 20 carbon atoms, an alkoxy group, an aryloxy group, said alkyl and aryl radicals being optionally substituted;
the amines of formula NR₇R₈R₉ in which R₇, R₈ and R₉, which may be identical or different, represent a hydrogen atom, an alkyl radical containing 1 to 12 carbon atoms or an aryl radical containing from 6 to 20 carbon atoms, said alkyl and aryl radicals being optionally substituted;
the amides of formula R₇CO—NR₈R₉ in which R₇, R₈ and R₉, which may be identical or different, represent a hydrogen atom, an alkyl radical containing 1 to 12 carbon atoms or an aryl radical containing from 6 to 20 carbon atoms, said alkyl and aryl radicals being optionally substituted;
the N-heterocyclic carbenes optionally derived from an imidazolium salt, chosen from the 1,3-bis(2,6-diisopropylphenyl)-1H-imidazol-3-ium salts (also referred to as Pr), 1,3-bis(2,6-diisopropylphenyl)-4,5-dihydro-1H-imidazol-3-ium (also referred to as s-IPr), 1,3-bis(2,4,6-trimethylphenyl)-1H-imidazol-3-ium (also referred to as IMes), 1,3-bis(2,4,6-trimethylphenyl)-4,5-dihydro-1H-imidazol-3-ium (also referred to as s-IMes), 4,5-dichloro-1,3-bis(2,6-diisopropylphenyl)-1H-imidazol-3-ium (also referred to as Cl₂—IPr), 1,3-di-tert-butyl-1H-imidazol-3-ium (also referred to as ItBu), and 1,3-di-tert-butyl-4,5-dihydro-1H-imidazol-3-ium (also referred to as s-ItBu), said salts being in the form of chloride or tetraphenylborate salts;
cyclopentadienyl and its derivatives selected from 1,2,3,4,5-pentakis(methyl)cyclopentadienyl, 1,2,3,4,5-pentakis(1-methylethyl)cyclopentadienyl and 1,2,3,4,5-pentakis(1-dimethylethyl)cyclopentadienyl;
the nitriles of formula NCR₁₀ in which R₁₀ represents an alkyl radical containing 1 to 12 carbon atoms or an aryl radical containing 6 to 20 carbon atoms, said alkyl and aryl radicals being optionally substituted;
the pyridine and its derivatives of formula (IV)

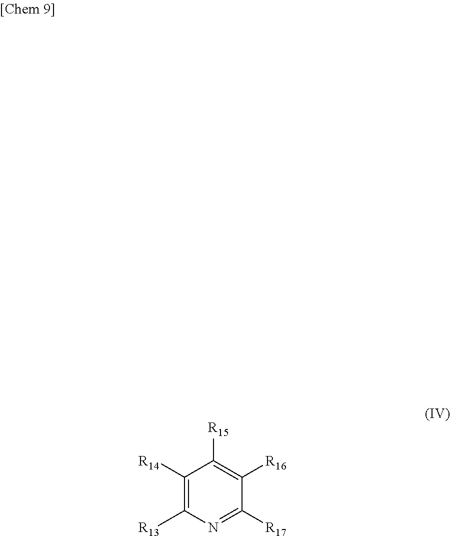

in which R₁₃, R₁₄, R₁₅, R₁₆ and R₁₇, which may be identical or different, represent a hydrogen atom, an alkyl radical containing 1 to 12 carbon atoms or an aryl radical containing 6 to 20 carbon atoms, said alkyl and aryl radicals being optionally substituted;
the bipyridines of formula (V)

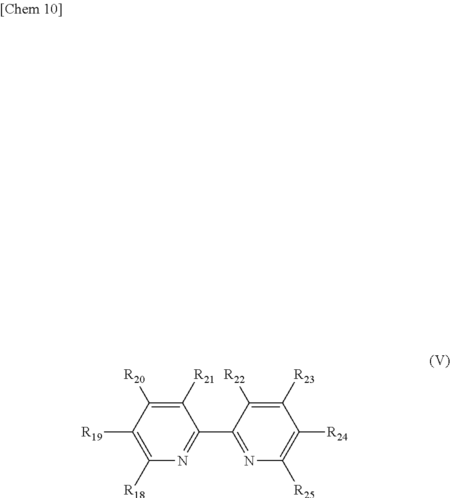

in which R₁₈, R₁₉, R₂₀, R₂₁, R₂₂, R₂₃, R₂₄ and R₂₅, which may be identical or different, represent a hydrogen atom, an alkyl radical containing 1 to 12 carbon atoms or an aryl radical containing 6 to 20 carbon atoms, said alkyl and aryl radicals being optionally substituted;
M represents Co, Fe, Mn, Rh, Ru, Pd; M representing in particular Co or Pd;
X represents a halide selected from F⁻, Cl⁻, Br⁻, I⁻; a trifluoromethylsulphonate (or triflate or CF₃SO₃⁻); an acetylacetonate (or acac or C₅H₇O₂⁻); an acetate (or CH₃COO⁻ or AcO⁻); a trifluoroacetate (or CF₃COO⁻ or TFAcO⁻);
a=0 to 12;
b=0 to 3;
c=0 to 12;
d=0 to 12;
in one or a mixture of at least two solvents chosen from:
the aromatic hydrocarbons chosen from benzene and toluene,
the nitriles chosen from acetonitrile, propionitrile and benzonitrile,
the linear or cyclic ethers chosen from diethyl ether, methyl vinyl ether, 1,1-diethoxyethane, dimethoxyethane, THF, methyltetrahydrofuran, tetrahydropyran and dioxane,
the esters such as ethyl acetate,
the carbonate esters such as propylene carbonate,
the ketones chosen from acetone and butanone,
the alkyl halides chosen from chloroform and methylene chloride,
the aryl halides chosen from chlorobenzene and dichlorobenzene;
   at a temperature of 110° C. or less.

When R₁, R₂ and R₃ are identical and represent a hydrogen atom, the cyclic anhydride of formula (I) is succinic anhydride and the acid of formula (II) is acrylic acid.

When R₁ represents a methyl radical and R₂ and R₃ are identical and represent a hydrogen atom; or R₃ represents a methyl radical and R₁ and R₂ are identical and represent a hydrogen atom, the cyclic anhydride of formula (I) is methyl succinic anhydride and the acid of formula (II) is crotonic acid.

When R₂ represents a methyl radical and R₁ and R₃ are identical and represent a hydrogen atom, the cyclic anhydride of formula (I) is methyl succinic anhydride and the acid of formula (II) is methacrylic acid.

When R₁ represents an ethyl radical, and R₂ and R₃ are identical and represent a hydrogen atom; or R₃ represents an ethyl radical, and R₁ and R₂ are identical and represent a hydrogen atom, the cyclic anhydride of formula (I) is ethyl succinic anhydride and the acid of formula (II) is penten-2-oic acid.

The method describes a new route for synthesising cyclic anhydrides of formula (I), in particular succinic anhydride, by carbonylating an acid of formula (II), in particular acrylic acid, with reagents that can be bio-sourced.

Indeed, when the acid of formula (II) is acrylic acid, although its main route of access is currently the oxidation of petroleum-based propylene, it can also be bio-sourced as described by Corma, A., Iborra, S., and Velty, A. (Chemical reviews, 2007, 107(6), 2411-2502 (doi:10.1021/cr050989d)), as shown schematically in FIG. 4.

In addition, the method of the invention is designed to be easily applied at industrial level and to be competitive compared with methods already implemented on an industrial scale, since it implements carbonyl metal complexes, already tried and tested in industrial hydroformylation methods, which can be used here under relatively mild pressure and temperature conditions (such as, for example, less than 20 bar and less than 100° C.).

The method of the invention therefore offers advantages over the synthesis method most commonly used in the industry, which is the hydrogenation of maleic anhydride, a petroleum-based compound, under high temperature conditions.

To date, there has been little development in the use of acrylic acid under carbonylation conditions. The inventors have noted that no direct hydroformylation reaction of acrylic acid has been described to date. In the closest examples of hydroformylation, the carboxylic acid function of acrylic acid was protected by a polymeric support (Dessole, G., Marchetti, M., & Taddei, M., Journal of combinatorial chemistry, 2003, 5(3), 198-200 (doi:10.1021/cc020055s)); Marchetti M., Botteghi C., Taddei M., Adv. Synth. Catal. 2003, 345, 1229-1236 (doi:10.1002/adsc.200303053)).

In 1962, following the development of his cyclising carbonylation reaction for acrylamide, J. Falbe sought to apply the same type of reaction to acrylic acid and its derivatives, and explains that this was not possible because: “The acrylic and methacrylic acids polymerise under the reaction conditions generally applied in a cyclisation reaction. The long-chain α,β-unsaturated acids give lactones via the migration of their double bond followed by the intramolecular addition of the carboxyl group onto this double bond.” (Falbe J., Ring closure with Carbon Monoxide. 1970, In Carbon Monoxide in Organic Synthesis (pp. 147-174). Springer, Berlin, Heidelberg).

In 1969, J. Tsuji tested the carbonylation of acrylic acid, catalysed by cobalt carbonyl, to determine whether acrylic acid was an intermediate in the carbonylation of propiolactone to succinic anhydride, but observed no trace of succinic anhydride at the end of catalysis. On the basis of this experience, Tsuji ruled out the possibility of acrylic acid being an intermediate in the synthesis of succinic anhydride from propiolactone, suggesting that this type of reaction condition would probably not allow the carbonylation of acrylic acid to give succinic anhydride. (Mori, Y.; Tsuji, J.; J. Bull. Chem. Soc. Jpn. 1969, 42 (3), 777-779 (doi:10.1246/bcsj.42.777)).

The carbonylation of acrylic acid therefore highlights two technical difficulties, namely the tendency of acrylic acid to polymerise under carbonylation conditions (Falbe), or even its total lack of reactivity (Tsuji).

For the purposes of this invention, “alkyl” means a linear or branched, saturated, optionally substituted carbon radical comprising 1 to 25 carbon atoms, for example 1 to 12 carbon atoms, for example 1 to 8 carbon atoms, for example 1 to 6 carbon atoms. Examples of saturated, linear or branched alkyl are methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, icosyl, henicosyl, docosyl, tricosyl, tetracosyl, pentacosyl radicals, and branched isomers thereof.

For the purposes of this invention, “cycloalkyl” or “cyclic alkyl” means a saturated, optionally substituted, cyclic carbon radical comprising 3 to 12 carbon atoms, for example 3 to 10 carbon atoms, for example 3 to 8 carbon atoms. Cycloalkyl radicals include cyclopropyl, cyclobutyl, cyclopentyl, cyclohexyl, cycloheptyl, cyclooctyl, bicyclo[2,1,1]hexyl, bicyclo[2,2,1]heptyl and adamantyl.

The term “aryl” refers to a mono- or poly-cyclic aromatic substituent containing from 6 to 20 carbon atoms. The aryl group may comprise, for example, 6 to 10 carbon atoms. Examples include phenyl, benzyl, naphthyl, binaphthyl, phenanthrenyl, pyrenyl, anthrancenyl, o-tolyl, m-tolyl, p-tolyl, mesityl and p-nitrophenyl, o-methoxyphenyl, m-methoxyphenyl and p-methoxyphenyl, o-methoxybenzyl, p-methoxybenzyl, m-methoxybenzyl, o-methylbenzyl, p-methylbenzyl and m-methylbenzyl.

The term “alkoxy” means an alkyl radical or a cycloalkyl radical, as defined above, linked by an oxygen atom (—O-alkyl or —O-cycloalkyl).

The term “aryloxy” means an aryl radical, as defined above, linked by an oxygen atom (—O-aryl).

“Halogen” means an atom selected from fluorine, chlorine, bromine and iodine. The term “halide” corresponds to the corresponding anion, i.e. F⁻, Cl⁻, Br⁻ and I⁻.

The term “phosphine” means a molecule of formula PR₄R₅R₆, in which R4, R5 and R6, which may be identical or different, represent a hydrogen atom, an alkyl radical containing 1 to 12 carbon atoms, an aryl radical containing 6 to 20 carbon atoms, an alkoxy group, an aryloxy group or a ferrocenyl group, said alkyl, aryl and ferrocenyl radicals being optionally substituted.

The term “polyphosphine” means a phosphine as defined in the context of the present invention, in which at least one of the substituents R₄, R₅ and R₆ is, in turn, substituted by one or several phosphinyl groups of formula —PR₁₁R₁₂, in which R₁₁ and R₁₂, which may be identical or different, represent an alkyl radical containing from 1 to 12 carbon atoms, an aryl radical containing from 6 to 20 carbon atoms, an alkoxy group or an aryloxy group, said alkyl, aryl and ferrocenyl radicals being optionally substituted. Examples include 1,2-bis(dimethylphosphino)ethane (dmpe), 1,2-bis(diphenylphosphino)ethane (dppe), 1,3-bis(diphenylphosphino)propane (dppp), 1,2-bis(dicyclohexylphosphino)ethane (dcpe), 1,2-bis(diphenylphosphino)benzene, 1,2-bis(diphenylphosphinomethyl)benzene, tris-[2-(diphenylphosphino)ethyl]phosphine, (9,9-dimethyl-9H-xanthene-4,5-diyl)bis(diphenylphosphane) (xantphos), bis(dicyclohexylphosphino)ferrocene (dcpf), bis(diphenylphosphino)ferrocene (dppf), and bis(diisopropylphosphino)ferrocene.

By “amine” is meant a group of formula NR₇R₈R₉ in which R₇, R₈ and R₉, which may be identical or different, represent a hydrogen atom, an alkyl radical containing from 1 to 12 carbon atoms, or an aryl radical containing from 6 to 20 carbon atoms, with the alkyl and aryl radicals being as defined in the context of the present invention. The alkyl and aryl radicals are optionally substituted. Examples include triethylamine, trimethylamine, N-diisopropylethylamine (DIPEA), diethylisopropylamine (DIEA) and diisopropylamine (DIPA).

By “amide” is meant a group of formula R₇CO—NR₈R₉ in which R₇, R₈ and R₉, which may be identical or different, represent a hydrogen atom, an alkyl radical containing 1 to 12 carbon atoms or an aryl radical containing 6 to 20 carbon atoms, said alkyl and aryl radicals being optionally substituted. Examples include N,N-dimethylformamide (or DMF).

In the context of the present invention, “cyclopentadienyl” represents —C₅H₅, and its derivatives are chosen from 1,2,3,4,5-pentakis(methyl)cyclopentadienyl, 1,2,3,4,5-pentakis(1-methylethyl)cyclopentadienyl and 1,2,3,4,5-pentakis(1-dimethylethyl)cyclopentadienyl.

By “nitrile” is meant a molecule of the formula NCR₁₀ in which R₁₀ represents an alkyl radical containing 1 to 12 carbon atoms or an aryl radical containing 6 to 20 carbon atoms, said alkyl and aryl radicals being optionally substituted. Examples include acetonitrile, propionitrile and benzonitrile.

By “ferrocenyl” group is meant a compound in which the iron is surrounded by two optionally substituted cyclopentadienyl rings of formula Fe[(C₅(R₂₇R₂₈R₂₉R₃₀R₃₁))(C₅(R₃₂R₃₃R₃₄R₃₅R₃₆))] in which R₂₇, R₂₈, R₂₉, R₃₀, R₃₁, R₃₂, R₃₃, R₃₄, R₃₅ and R₃₆, which may be identical or different, represent a hydrogen atom, an alkyl radical containing from 1 to 12 carbon atoms or an aryl radical containing from 6 to 20 carbon atoms, said alkyl and aryl radicals being optionally substituted. Examples of ferrocenyl groups include ferrocene (Fe(C₅H₅)₂), methylferrocene, 1,2,4,1′,2′,4′-hexamethylferrocene, t-butylferrocene and phenylferrocene.

“Pyridine” represents C₅H₅N and its derivatives are molecules of formula (IV)

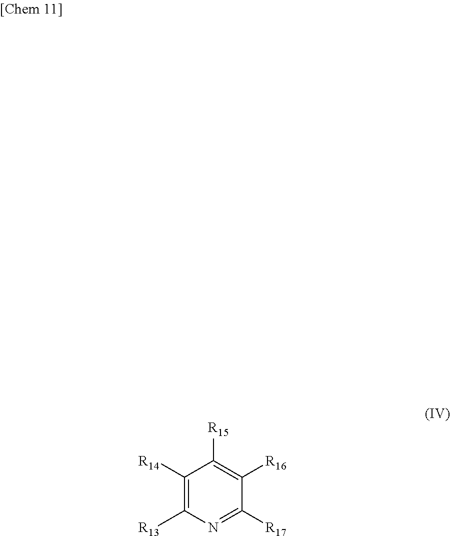

in which R₁₃, R₁₄, R₁₅, R₁₆ and R₁₇, which may be identical or different, represent a hydrogen atom, an alkyl radical containing 1 to 12 carbon atoms or an aryl radical containing 6 to 20 carbon atoms, said alkyl and aryl radicals being optionally substituted. The pyridine derivatives are, for example, selected from 2-methylpyridine, 3-methylpyridine, 4-methylpyridine, 2,3-dimethylpyridine, 2,4-dimethylpyridine, 2,5-dimethylpyridine, 2,6-dimethylpyridine, 3,4-dimethylpyridine, 3,5-dimethylpyridine, 2,3,4-trimethylpyridine, 2,3,5-trimethylpyridine, 2,3,6-trimethylpyridine, 2,4,5-trimethylpyridine, 2,4,6-trimethylpyridine, 3,4,5-trimethylpyridine, 2,3,4,5-tetramethylpyridine, 2,3,4,6-tetramethylpyridine, 2,3,5,6-tetramethylpyridine.

“Bipyridines” are molecules of formula (V)

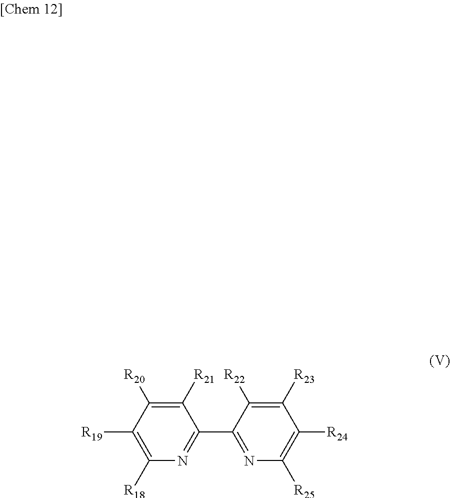

in which R₁₈, R₁₉, R₂₀, R₂₁, R₂₂, R₂₃, R₂₄ and R₂₅, which may be identical or different, represent a hydrogen atom, an alkyl radical containing 1 to 12 carbon atoms or an aryl radical containing 6 to 20 carbon atoms, said alkyl and aryl radicals being optionally substituted. Examples include 2,2′-bipyridine, 3,3′-dimethyl-2,2′-bipyridine, 4,4′-dimethyl-2,2′-bipyridine, 5,5′-dimethyl-2,2′-bipyridine and 6,6′-dimethyl-2,2′-bipyridine.

The term “carboxylate” designates a group of formula —OC(O)R₂₆, in which R₂₆ is chosen from a hydrogen atom, an alkyl radical containing 1 to 12 carbon atoms, an aryl radical containing 6 to 20 carbon atoms, with the alkyl and aryl radicals being defined within the scope of the present invention.

The alkyl and aryl radicals are optionally substituted.

The term “carbonyl” refers to a group of formula —C(O)R, in which R is selected from an alkyl radical containing from 1 to 12 carbon atoms, an aryl radical containing from 6 to 10 carbon atoms, together with alkyl and aryl radicals as defined within the scope of the present invention. The alkyl and aryl radicals are optionally substituted.

The alkyl, aryl and ferrocenyl radicals may optionally be substituted by one or more hydroxyl groups (—OH), one or more alkoxy groups (—O-alkyl); one or more aryloxy groups (—O-aryl); one or more halogen atoms chosen from fluorine, chlorine, bromine and iodine atoms; one or more nitro groups (—NO₂); one or more nitrile groups (—CN); one or more carbonyl groups (—C(O)R); one or more carboxylate groups (—OC(O)R₂₆); one or more amine groups (NR₇R₈R₉); one or more amide groups (R₇CO—NR₈R₉); one or more alkyl radicals; one or more aryl radicals; with alkyl, aryl and all groups as defined within the scope of the present invention.

According to a first embodiment, R₁, R₂ and R₃, which may be identical or different, represent a hydrogen atom, an alkyl radical containing from 1 to 25 carbon atoms, an aryl radical containing from 6 to 20 carbon atoms, a hydroxyl group, an alkoxy group or an aryloxy group, said alkyl and aryl radicals being optionally substituted.

Said alkyl and aryl radicals are optionally substituted by one or more hydroxyl groups (—OH), one or more alkoxy groups (—O-alkyl); one or more aryloxy groups (—O-aryl); one or more halogen atoms chosen from fluorine, chlorine, bromine and iodine atoms; one or more nitro groups (—NO₂); one or more nitrile groups (—CN); one or more carbonyl groups (—C(O)R); one or more carboxylate groups (—OC(O)R₂₆); one or more amine groups (NR₇R₈R₉); one or more amide groups (R₇CO—NR₈R₉); one or more alkyl radicals; one or more aryl radicals; with alkyl, aryl and all the groups as defined within the scope of the present invention.

According to a second embodiment, R₁, R₂ and R₃, which may be identical or different, represent a hydrogen atom, an alkyl radical containing 1 to 25 carbon atoms, chosen from methyl, ethyl, propyl, butyl, pentyl, hexyl heptyl, octyl, nonyl, decyl, undecyl, dodecyl, tridecyl, tetradecyl, pentadecyl, hexadecyl, heptadecyl, octadecyl, nonadecyl, icosyl, henicosyl, docosyl, tricosyl, tetracosyl, pentacosyl and branched isomers thereof; an aryl group containing 6 to 10 carbon atoms, selected from phenyl, benzyl and naphthyl.

Said alkyl and aryl radicals are optionally substituted by one or more hydroxyl groups (—OH), one or more alkoxy groups (—O-alkyl); one or more aryloxy groups (—O-aryl); one or more halogen atoms chosen from fluorine, chlorine, bromine and iodine atoms; one or more nitro groups (—NO₂); one or more nitrile groups (—CN); one or more carbonyl groups (—C(O)R); one or more carboxylate groups (—OC(O)R₂₆); one or more amine groups (NR₇R₈R₉); one or more amide groups (R₇CO—NR₈R₉); one or more alkyl radicals; one or more aryl radicals; with alkyl, aryl and all the groups as defined within the scope of the present invention.

According to another embodiment, R₁, R₂ and R₃, which may be identical or different, represent a hydrogen atom, an alkyl radical containing 1 to 12 carbon atoms, for example 1 to 8 carbon atoms, for example 1 to 6 carbon atoms, chosen in particular from methyl, ethyl, propyl, butyl, pentyl and hexyl radicals, and their branched isomers, in particular from methyl, ethyl and propyl radicals, more particularly from methyl and ethyl radicals.

According to another embodiment, R₃ represents a hydrogen atom, and R₁ and R₂, which may be identical or different, represent a hydrogen atom, an alkyl radical containing 1 to 12 carbon atoms, for example 1 to 8 carbon atoms, for example 1 to 6 carbon atoms, chosen in particular from methyl, ethyl, propyl, butyl, pentyl and hexyl radicals, and their branched isomers, in particular from methyl, ethyl and propyl radicals, more particularly from methyl and ethyl radicals.

According to another embodiment, R₃ represents a hydrogen atom, one of R₁ and R₂ represents a hydrogen atom, and the other of R₁ and R₂ represents a hydrogen atom, an alkyl radical containing 1 to 12 carbon atoms, for example 1 to 8 carbon atoms, for example 1 to 6 carbon atoms, in particular chosen from methyl, ethyl, propyl, butyl, pentyl and hexyl radicals, and their branched isomers, in particular from methyl, ethyl and propyl radicals, more particularly from methyl and ethyl radicals.

According to a third embodiment, R₁ represents a methyl radical, and R₂ and R₃ are identical and represent a hydrogen atom.

According to a fourth embodiment, R₃ represents a methyl radical, and R₁ and R₂ are identical and represent a hydrogen atom.

According to a fifth embodiment, R₁, R₂ and R₃ are identical and represent a hydrogen atom.

According to another embodiment, R₂ represents a methyl radical, and R₁ and R₃ are identical and represent a hydrogen atom.

According to another embodiment, R₁ represents an ethyl radical, and R₂ and R₃ are identical and represent a hydrogen atom.

In all the embodiments and variants of the invention, the method of the invention takes place in the presence of a metal catalyst of formula (III) as defined above.

In a sixth embodiment of the invention, the metal catalyst is of formula (III)

[Chem 13]

LₐM_{b}X_{c}(CO)_{d} (III)
wherein
L represents Na⁺, K⁺, Li⁺, Cs⁺, Mg²⁺, Ca²⁺, NH₄⁺, [((C₆H₅)₃P)₂N]⁺; a ligand chosen from:
   the phosphines of formula PR₄R₅R₆, in which R₄, R₅ and R₆, which may be identical or different, represent a hydrogen atom, an alkyl radical containing 1 to 12 carbon atoms, an aryl radical containing from 6 to 20 carbon atoms, an alkoxy group, an aryloxy group or a ferrocenyl group, said alkyl, aryl and ferrocenyl radicals being optionally substituted;
   the polyphosphines which mean a phosphine as defined above, in which at least one of the substituents R₄, R₅ and R₆ is substituted by one or several phosphinyl groups of formula —PR₁₁R₁₂, in which R₁₁ and R₁₂, which may be identical or different, represent an alkyl radical containing 1 to 12 carbon atoms, an aryl radical containing from 6 to 20 carbon atoms, an alkoxy group, an aryloxy group, said alkyl and aryl radicals being optionally substituted;
   amines of formula NR₇R₈R₉ in which R₇, R₈ and R₉, which may be identical or different, represent an alkyl radical containing 1 to 12 carbon atoms, an aryl radical containing from 6 to 20 carbon atoms, said alkyl and aryl radicals being optionally substituted;
   the N-heterocyclic carbenes optionally derived from an imidazolium salt, chosen from the 1,3-bis(2,6-diisopropylphenyl)-1H-imidazol-3-ium salts (also referred to as Pr), 1,3-bis(2,6-diisopropylphenyl)-4,5-dihydro-1H-imidazol-3-ium (also referred to as s-IPr), 1,3-bis(2,4,6-trimethylphenyl)-1H-imidazol-3-ium (also referred to as IMes), 1,3-bis(2,4,6-trimethylphenyl)-4,5-dihydro-1H-imidazol-3-ium (also referred to as s-IMes), 4,5-dichloro-1,3-bis(2,6-diisopropylphenyl)-1H-imidazol-3-ium (also referred to as Cl₂—IPr), 1,3-di-tert-butyl-1H-imidazol-3-ium (also referred to as ItBu), and 1,3-di-tert-butyl-4,5-dihydro-1H-imidazol-3-ium (also referred to as s-ItBu), said salts being in the form of chloride or tetraphenylborate salts;
   nitriles of formula NCR₁₀ in which R₁₀ represents an alkyl radical containing 1 to 12 carbon atoms or an aryl radical containing 6 to 20 carbon atoms, said alkyl and aryl radicals being optionally substituted;
   the pyridine and its derivatives of formula (IV)

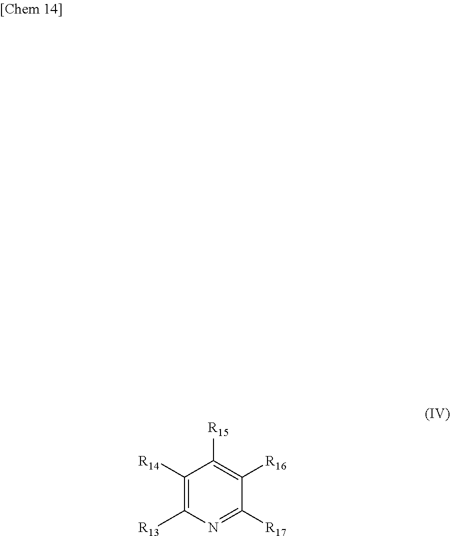

in which R₁₃, R₁₄, R₁₅, R₁₆ and R₁₇, which may be identical or different, represent a hydrogen atom, an alkyl radical containing 1 to 12 carbon atoms or an aryl radical containing 6 to 20 carbon atoms, said alkyl and aryl radicals being optionally substituted;
   the bipyridines of formula (V)

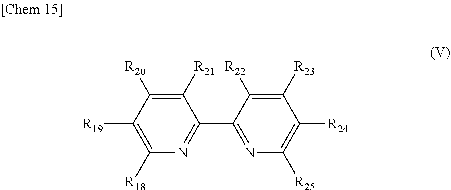

in which R₁₈, R₁₉, R₂₀, R₂₁, R₂₂, R₂₃, R₂₄ and R₂₅, which may be identical or different, represent a hydrogen atom, an alkyl radical containing 1 to 12 carbon atoms or an aryl radical containing 6 to 20 carbon atoms, said alkyl and aryl radicals being optionally substituted;
M represents Co, Fe, Rh, Pd; M representing in particular Co or Pd;
X represents a halide selected from F⁻, Cl⁻, Br⁻, I⁻; a trifluoromethylsulphonate (or triflate or CF₃SO₃⁻); an acetylacetonate (or acac or C₅H₇O₂⁻); an acetate (or CH₃COO⁻ or AcO⁻); a trifluoroacetate (or CF₃COO⁻ or TFAcO⁻);
a=0 to 12;
b=0 to 3;
c=0 to 12;
d=0 to 12.

In a seventh embodiment of the invention, the metal catalyst is of formula (III)

[Chem 16]

LₐM_{b}X_{c}(CO)_{d} (III)
wherein
L represents Na⁺, K⁺, NH₄⁺, [((C₆H₅)₃P)₂N]⁺; a ligand chosen from
   the phosphines of formula PR₄R₅R₆, in which R₄, R₅ and R₆, which may be identical or different, represent a hydrogen atom, an alkyl radical containing 1 to 12 carbon atoms, an aryl radical containing from 6 to 20 carbon atoms, an alkoxy group, an aryloxy group or a ferrocenyl group, said alkyl, aryl and ferrocenyl radicals being optionally substituted;
   the polyphosphines which mean a phosphine as defined above, in which at least one of the substituents R₄, R₅ and R₆ is substituted by one or several phosphinyl groups of formula —PR₁₁R₁₂, in which R₁₁ and R₁₂, which may be identical or different, represent an alkyl radical containing 1 to 12 carbon atoms, an aryl radical containing from 6 to 20 carbon atoms, an alkoxy group, an aryloxy group, said alkyl and aryl radicals being optionally substituted;
   the N-heterocyclic carbenes optionally derived from an imidazolium salt, chosen from the 1,3-bis(2,6-diisopropylphenyl)-1H-imidazol-3-ium salts (also referred to as Pr), 1,3-bis(2,6-diisopropylphenyl)-4,5-dihydro-1H-imidazol-3-ium (also referred to as s-IPr), 1,3-bis(2,4,6-trimethylphenyl)-1H-imidazol-3-ium (also referred to as IMes), 1,3-bis(2,4,6-trimethylphenyl)-4,5-dihydro-1H-imidazol-3-ium (also referred to as s-IMes), 4,5-dichloro-1,3-bis(2,6-diisopropylphenyl)-1H-imidazol-3-ium (also referred to as Cl₂—IPr), 1,3-di-tert-butyl-1H-imidazol-3-ium (also referred to as ItBu), and 1,3-di-tert-butyl-4,5-dihydro-1H-imidazol-3-ium (also referred to as s-ItBu), said salts being in the form of chloride or tetraphenylborate salts;
   the pyridine and its derivatives of formula (IV)

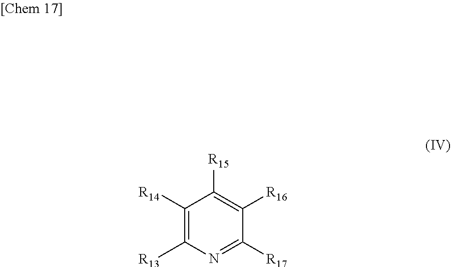

in which R₁₃, R₁₄, R₁₅, R₁₆ and R₁₇, which may be identical or different, represent a hydrogen atom, an alkyl radical containing 1 to 12 carbon atoms or an aryl radical containing 6 to 20 carbon atoms, said alkyl and aryl radicals being optionally substituted;
   the bipyridines of formula (V)

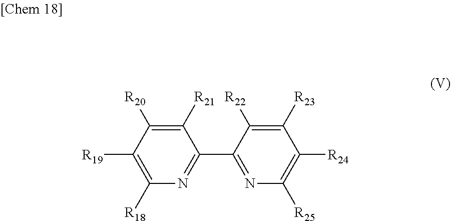

in which R₁₈, R₁₉, R₂₀, R₂₁, R₂₂, R₂₃, R₂₄ and R₂₅, which may be identical or different, represent a hydrogen atom, an alkyl radical containing 1 to 12 carbon atoms or an aryl radical containing 6 to 20 carbon atoms, said alkyl and aryl radicals being optionally substituted;
M represents Co or Pd; in particular, M represents Co;
X represents a halide selected from Cl⁻, Br⁻, acetylacetonate (or acac or C₅H₇O₂⁻); acetate (or CH₃COO⁻ or AcO⁻); trifluoroacetate (or CF₃COO⁻ or TFAcO⁻);
a=0 to 8;
b=1 to 2;
c=0 to 8;
d=0 to 8.

In another embodiment, d=0.

In another embodiment, X represents an acetylacetonate (or acac or C₅H₇O₂⁻); an acetate (or CH₃COO⁻ or AcO⁻); a trifluoroacetate (or CF₃COO⁻ or TFAcO⁻), c being non-zero (c≠0).

In another embodiment, d=0, and X represents an acetylacetonate (or acac or C₅H₇O₂⁻); an acetate (or CH₃COO⁻ or AcO⁻); a trifluoroacetate (or CF₃COO⁻ or TFAcO⁻), c being non-zero (c≠0).

In another embodiment, M is Pd.

In another embodiment, L is a ligand as defined above.

In another embodiment, M is Pd and L is a ligand as defined above.

In another embodiment, M is Pd, L is a ligand as defined above, d=0, and X represents an acetylacetonate (or acac or C₅H₇O₂⁻); an acetate (or CH₃COO⁻ or AcO⁻); a trifluoroacetate (or CF₃COO⁻ or TFAcO⁻), c being non-zero (c≠0).

In an eighth embodiment, the metal catalyst is CO₂(CO)₈, NaCo(CO)₄, or [(C₆H₅)₃P)₂N]Co(CO)₄.

In another embodiment, the metal catalyst is Pd(CF₃COO)₂.

In all the embodiments and variants of the invention, the amount of metal catalyst of formula (III) is from 0.1 to 10 mol %, preferably from 0.5 to 8 mol %, more preferably from 1 to 6 mol %, relative to the amount of acid of formula (II).

The metal catalysts of formula (III) can, if required, be immobilised on heterogeneous supports, for example, in order to ensure easy separation and/or recycling of said catalyst. Said heterogeneous supports can be chosen from the supports based on silica gel or plastic polymers such as, for example, polystyrene; the carbon supports chosen from carbon nanotubes; silica carbide; alumina; titanium dioxide (TiO₂) or magnesium chloride (MgCl₂).

The method of the invention can also take place in the presence of an additive or a ligand to improve the performance of the method.

In the context of the invention, “additive” refers to a compound capable of improving the reactivity of the reagents for converting the acid of formula (II) into the cyclic anhydride of formula (I).

By ligand we mean a molecule carrying chemical functions allowing it to bind to the metal centre of the catalyst to form a coordination complex, thereby improving the catalytic power of the catalyst.

In a ninth embodiment, the method of the invention takes place in the presence of an additive of formula ZY in which
Y represents an anion selected from F⁻, C⁻, Br⁻, I⁻; and
Z represents a cation chosen from Na⁺, K⁺, Li⁺, Cs⁺, Mg²⁺, Ca²⁺, NH₄⁺; and/or
   of a ligand chosen from:
the phosphines of formula PR₄R₅R₆, in which R₄, R₅ and R₆, which may be identical or different, represent a hydrogen atom, an alkyl radical containing 1 to 12 carbon atoms, an aryl radical containing from 6 to 20 carbon atoms, an alkoxy group, an aryloxy group or a ferrocenyl group, said alkyl, aryl and ferrocenyl radicals being optionally substituted;
the polyphosphines which mean a phosphine as defined above, in which at least one of the substituents R₄, R₅ and R₆ is substituted by one or several phosphinyl groups of formula —PR₁₁R₁₂, in which R₁₁ and R₁₂, which may be identical or different, represent an alkyl radical containing 1 to 12 carbon atoms, an aryl radical containing from 6 to 20 carbon atoms, an alkoxy group, an aryloxy group, said alkyl and aryl radicals being optionally substituted;
the amines of formula NR₇R₈R₉ in which R₇, R₈ and R₉, which may be identical or different, represent a hydrogen atom, an alkyl radical containing 1 to 12 carbon atoms or an aryl radical containing from 6 to 20 carbon atoms, said alkyl and aryl radicals being optionally substituted;
the amides of formula R₇CO—NR₈R₉ in which R₇, R₈ and R₉, which may be identical or different, represent a hydrogen atom, an alkyl radical containing 1 to 12 carbon atoms or an aryl radical containing from 6 to 20 carbon atoms, said alkyl and aryl radicals being optionally substituted;
the N-heterocyclic carbenes optionally derived from an imidazolium salt, chosen from the 1,3-bis(2,6-diisopropylphenyl)-1H-imidazol-3-ium salts (also referred to as Pr), 1,3-bis(2,6-diisopropylphenyl)-4,5-dihydro-1H-imidazol-3-ium (also referred to as s-IPr), 1,3-bis(2,4,6-trimethylphenyl)-1H-imidazol-3-ium (also referred to as IMes), 1,3-bis(2,4,6-trimethylphenyl)-4,5-dihydro-1H-imidazol-3-ium (also referred to as s-IMes), 4,5-dichloro-1,3-bis(2,6-diisopropylphenyl)-1H-imidazol-3-ium (also referred to as Cl₂—IPr), 1,3-di-tert-butyl-1H-imidazol-3-ium (also referred to as ItBu), and 1,3-di-tert-butyl-4,5-dihydro-1H-imidazol-3-ium (also referred to as s-ItBu), said salts being in the form of chloride or tetraphenylborate salts;
cyclopentadienyl and its derivatives selected from 1,2,3,4,5-pentakis(methyl)cyclopentadienyl, 1,2,3,4,5-pentakis(1-methylethyl)cyclopentadienyl and 1,2,3,4,5-pentakis(1-dimethylethyl)cyclopentadienyl;
the nitriles of formula NCR₁₀ in which R₁₀ represents an alkyl radical containing 1 to 12 carbon atoms or an aryl radical containing 6 to 20 carbon atoms, said alkyl and aryl radicals being optionally substituted;
the pyridine and its derivatives of formula (IV)

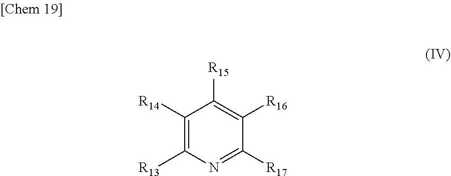

in which R₁₃, R₁₄, R₁₅, R₁₆ and R₁₇, which may be identical or different, represent a hydrogen atom, an alkyl radical containing 1 to 12 carbon atoms or an aryl radical containing 6 to 20 carbon atoms, said alkyl and aryl radicals being optionally substituted;
the bipyridines of formula (V)

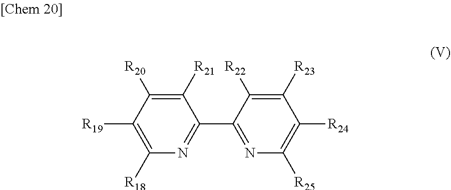

in which R₁₈, R₁₉, R₂₀, R₂₁, R₂₂, R₂₃, R₂₄ and R₂₅, which may be identical or different, represent a hydrogen atom, an alkyl radical containing 1 to 12 carbon atoms or an aryl radical containing 6 to 20 carbon atoms, said alkyl and aryl radicals being optionally substituted.

The additive can be KI, for example.

Preferably, the ligand is triphenylphosphine, tri(o-toly)phosphine, tri-n-butylphosphine, tri-tert-buty/phosphine, trimethylphosphite, triethylphosphite, tris(pentafluorophenyl)phosphine, diphenyl(pentafluorophenyl)phosphine, 1,2-bis(dimethylphosphino)ethane (dmpe), 1,2-bis(diphenylphosphino)ethane (dppe), 1,3-bis(diphenylphosphino)propane (dppp), 1,2-bis(dicyclohexylphosphino)ethane (dcpe); more preferably 1,2-bis(dicyclohexylphosphino)ethane, 1,2-bis(diphenylphosphino)ethane (dppe), 1,3-bis(diphenylphosphino)propane (dppp), even more preferably 1,2-bis(dicyclohexylphosphino)ethane.

The additives and the ligands can be selected and used independently of each other.

When an additive is used, the quantity of additive is from 0.5 to 15 molar equivalents relative to the metal catalyst of formula (III), preferably from 1 to 10 equivalents, more preferably from 1 to 5 equivalents.

When a ligand is used, the amount of ligand is:
by 1 molar equivalent relative to the metal catalyst of formula (III) when the ligand is a polyphosphine;
from 1 to 2 molar equivalents relative to the metal catalyst of formula (III) when the ligand is a phosphine, preferably 1 molar equivalent;
from 0.5 to 12 molar equivalents relative to the metal catalyst of formula (III) when the ligand is an amine, an amide, an N-heterocyclic carbene, cyclopentadienyl and its derivatives, nitriles, pyridine and its derivatives, bipyridines, preferably 1 to 6 molar equivalents, more preferably 1 to 4 molar equivalents.

As mentioned previously, the method of the invention can take place under pressure of carbon monoxide exclusively or of a mixture of carbon monoxide and dihydrogen.

In all variants and all embodiments of the invention, the method takes place under a total pressure of carbon monoxide (CO) or a mixture of carbon monoxide and dihydrogen (CO/H₂) of 5 to 60 bar, preferably between 5 and 50 bar, even more preferably between 10 and 20 bar.

When a mixture of carbon monoxide and hydrogen (CO/H₂) is used, the proportion of hydrogen is from 1 to 50%, preferably from 1 to 10%, and more preferably from 5% to 10%.

The method of the invention takes place at a temperature of between 4° and 110° C., preferably between 6° and 110° C., and more preferably between 8° and 90° C.

In all the embodiments and variants of the invention, the concentration of the acid of formula (II) is between 0.01 and 5 M, preferably between 0.05 and 2 M, more preferably between 0.1 and 2 M.

In all the embodiments and variants of the invention, the duration of the reaction depends on the rate of conversion of the acid of formula (II) into the cyclic anhydride of formula (I) and on the metal catalyst used. The reaction can last from 1 hour to 100 hours.

In all embodiments and variants of the invention, the method of the invention takes place in one or a mixture of at least two solvents preferably chosen from:
the aromatic hydrocarbons chosen from benzene and toluene,
the linear or cyclic ethers chosen from diethyl ether, methyl vinyl ether, 1,1-diethoxyethane, dimethoxyethane, THF, methyltetrahydrofuran, tetrahydropyran and dioxane,
the esters such as ethyl acetate,
the carbonate esters such as propylene carbonate,
the ketones chosen from acetone and butanone,
the alkyl halides chosen from chloroform and methylene chloride,
the aryl halides chosen from chlorobenzene and dichlorobenzene.

In a tenth embodiment, the method of the invention takes place

in one or a mixture of at least two solvents chosen from aromatic hydrocarbons chosen from benzene and toluene;
with a mixture of carbon monoxide and hydrogen (CO/H₂); and
in the presence of phosphine or polyphosphine ligands.

According to an eleventh embodiment of the invention, the method of the invention occurs with carbon monoxide or a mixture of carbon monoxide and hydrogen (CO/H₂);

in one or a mixture of solvents selected from nitriles, in particular selected from acetonitrile, propionitrile and benzonitrile; and
in the absence of ligand.

The various reagents, catalysts, additives, ligands and solvents used directly or indirectly in the method of the invention are, in general, commercial compounds/solvents or can be prepared by methods already described in the literature and known to the person skilled in the art.

Another object of the invention is the use of a method according to the invention in the manufacture of food additives, plasticisers, polymers of interest, in particular polyurethanes and elastanes, resins, coatings and pharmaceutical products.

The method for manufacturing food additives, plasticisers, polymers of interest, in particular polyurethanes and elastanes, resins, coatings and pharmaceutical products, can comprise a step of preparing a cyclic anhydride of formula (I), in particular succinic anhydride, by the method of the invention.

### EXAMPLES

The various reagents and solvents used in the method of the invention and in the examples are, in general, commercial compounds or can be prepared by any method known to the person skilled in the art.

Unless specified, all reactions were carried out under argon, in an anhydrous and inert atmosphere (<5 ppm of H₂O and of O₂) using conventional techniques in a schlenk with a vacuum ramp or in a glove box (mBraun LabMaster DP). The glassware was dried for several hours at 120° C. in an oven before use. The reaction solvents were dried by conventional methods, distilled and stored in an inert atmosphere on 4 Å molecular sieve. The 4 Å molecular sieve (Aldrich) was dried under dynamic vacuum at 250° C. for 48 h before use. All the products were purchased from standard suppliers (Sigma Aldrich, Strem, Alfa-Aesar, Acros, etc), and dried or degassed beforehand if necessary. The carbon monoxide comes from Air Products 4.7 purity pressurised cylinders; the hydrogen comes from Messer 5.2 purity pressurised cylinders.

The synthesised compounds were characterised and quantified using analytical and mass spectrometry techniques using the Shimadzu GCMS QP2010 Ultra instrument, equipped with a Supelco SLB-ms silica column (30 m×0.25 mm×0.25 μm). The carrier gas is helium (purity 6.0) from Messer. The products were identified by comparison with commercial standards and calibrated using mesitylene as an internal standard.

### Experimental Protocol:

1. Under an inert atmosphere, the catalyst (mol %), the ligand (mol %), the additive (mol %), the acrylic acid (mol/L) and the solvent are introduced into an autoclave that has been purged three times with a flow of the gas or gas mixture used. The autoclave contains a glass tube for collecting the reaction mixture and is equipped with a magnetic stirrer. In order to monitor the yield of succinic anhydride and the conversion of acrylic acid, an internal standard can be added. The order of introduction of the acrylic acid, solvent, internal standard, catalyst, ligand or additive has no influence on the reaction.
2. The autoclave is then sealed, and the gas or gas mixture is injected into the autoclave to the desired total pressure.
3. The autoclave is placed in a heating block under magnetic stirring.
4. The conversion to acrylic acid and the yield of succinic anhydride can be monitored using GC-MS.

The conversions and yields described were measured by GC-MS using an internal standard, mesitylene, which was added at the start of the reaction and for which a calibration curve had previously been produced.

### Example 1

Dicobalt octacarbonyl (17.1 mg; 0.05 mmol; 0.05 eq), toluene (1 mL), mesitylene (14 μL; 0.1 mmol; 0.1 eq) and acrylic acid (68 μL; 1 mmol; 1 eq) were introduced into the autoclave, which was purged three times with a flow of carbon monoxide. The autoclave is sealed and subjected to a pressure of 15 bar of carbon monoxide, then put under magnetic stirring for 6 hours in a heating block at 80° C. A conversion of 43% and a yield of 18% were obtained, with a selectivity of 42% in favour of succinic anhydride.

### Example 2

Dicobalt octacarbonyl (17.1 mg; 0.05 mmol; 0.05 eq), potassium iodide (8.3 mg; 0.05 mmol; 0.05 eq), toluene (1 mL), mesitylene (14 μL; 0.1 mmol; 0.1 eq) and acrylic acid (68 μL; 1 mmol; 1 eq) were introduced into the autoclave, which was purged three times with a flow of carbon monoxide. The autoclave is sealed and subjected to a pressure of 15 bar of carbon monoxide, then put under magnetic stirring for 6 hours in a heating block at 80° C. A conversion of 47% and a yield of 33% were obtained, with a selectivity of 70% in favour of succinic anhydride.

### Example 3

Dicobalt octacarbonyl (17.1 mg; 0.05 mmol; 0.05 eq), triphenylphosphane (13.1 mg; 0.05 mmol; 0.05 eq), toluene (1 mL), mesitylene (14 μL; 0.1 mmol; 0.1 eq) and acrylic acid (68 μL; 1 mmol; 1 eq) were introduced into the autoclave, which was purged three times with a flow of carbon monoxide. The autoclave is sealed and subjected to a pressure of 15 bar of carbon monoxide, then put under magnetic stirring for 6 hours in a heating block at 80° C. A conversion of 55% and a yield of 39% were obtained, with a selectivity of 71% in favour of succinic anhydride.

### Example 4

Dicobalt octacarbonyl (17.1 mg; 0.05 mmol; 0.05 eq), 1,2-bis(diphenylphosphino)ethane (19.9 mg; 0.05 mmol; 0.05 eq), toluene (1 mL), mesitylene (14 μL; 0.1 mmol; 0.1 eq) and acrylic acid (68 μL; 1 mmol; 1 eq) were introduced into the autoclave, which was purged three times with a carbon monoxide/hydrogen flow (95:5). The autoclave is sealed and subjected to a pressure of 16 bar of a mixture of carbon monoxide/dihydrogen (95:5), then put under magnetic stirring for 6 hours in a heating block at 80° C. A conversion of 78% and a yield of 63% were obtained, with a selectivity of 81% in favour of succinic anhydride.

### Example 5

Dicobalt octacarbonyl (17.1 mg; 0.05 mmol; 0.05 eq), 1,2-bis(dicyclohexylphosphino)ethane (21.1 mg; 0.05 mmol; 0.05 eq), toluene (2 mL), mesitylene (14 μL; 0.1 mmol; 0.1 eq) and acrylic acid (68 μL; 1 mmol; 1 eq) were introduced into the autoclave which was purged three times by a carbon monoxide/dihydrogen flow (95:5). The autoclave is sealed and subjected to a pressure of 16 bar of a mixture of carbon monoxide/dihydrogen (95:5), then put under magnetic stirring for 6 hours in a heating block at 90° C. A yield of 96% is obtained.

### Tests Carried Out:

The succinic anhydride yields and acrylic acid conversions were obtained by GC-MS, following a calibration with commercial samples of succinic anhydride and acrylic acid. The percentage values are given with an uncertainty of +/−5 percentage points. Some acrylic acid conversions could not be determined (nd=not determined), because the acrylic acid peak overlapped too much with that of toluene (in the case of low conversion) or that of propionic acid, a by-product (non-negligible quantity of propionic acid).

Given the uncertainty of our measurements, a yield is considered to be significantly higher or lower than another yield if its value is at least 5 percentage points higher or lower than the other yield. The selectivities in favour of succinic anhydride were calculated from the yield of succinic anhydride and the conversion of acrylic acid: the ratio of yield to conversion. Other formed by-products include propionic acid (observed in GC-MS) and probably acrylic acid oligomers.

The results of the various experiments are set out in the tables below.

TABLE 1
Optimisation of various reaction parameters in
high-pressure carbon monoxide reactions.
All the reactions were performed in toluene, with an
acrylic acid concentration of 1 mol/L, under a pressure of CO,
with a total pressure of 50 bar, with a reaction time of 18 h.

| Catalyst | Ligand | T | Succinic anhydride | |
|---|---|---|---|---|
| (mol %) | (mol %) | (° C.) | Yield (%) | Selectivity (%) |
| Co₂(CO)₈ | | 80 | 18 | nd |
| (5) | | | | |
| NaCo(CO)₄ | | 80 | 8 | 13 |
| (5) | | | | |
| PPNCo(CO)₄ | | 80 | 8 | 16 |
| (5) | | | | |
| Co₂(CO)₈ | Triphenyl- | 80 | 18 | 31 |
| (5) | phosphine | | | |
| | (5) | | | |
| Co₂(CO)₈ | Triphenyl- | 80 | 8 | 11 |
| (5) | phosphine | | | |
| | (10) | | | |
| Co₂(CO)₈ | | 110 | 22 | 88 |
| (5) | | | | |
| Co₂(CO)₈ | | 150 | 6 | 11 |
| (5) | | | | |

PPNCo(CO₎₄ = bis(triphenylphosphine)iminium tetracarbonylcobaltate

The results presented in [Table 1] describe experiments carried out at high pressure in carbon monoxide (50 bar) and allow comparison of different catalytic systems (catalyst and ligand).

Under 50 bar of carbon monoxide, Co₂(CO)₈ is a better catalyst than NaCo(CO)₄ and PPNCo(CO)₄ (PPN=bis(triphenylphosphine)iminium or [((C₆H₅)₃P)₂N]⁺).

Adding a triphenylphosphine ligand in an equimolar quantity to Co₂(CO)₈ does not increase yield; doubling the quantity of ligand has a deleterious effect on yield.

Raising the temperature from 80 to 110° C. does not significantly increase yield; raising the temperature to 150° C. has a deleterious effect on yield.

TABLE 2
Optimisation of the carbon monoxide pressure
All reactions were performed in toluene, with an acrylic
acid concentration of 1 mol/L, under CO pressure.

| | Total | | | Succinic anhydride | |
|---|---|---|---|---|---|
| Catalyst | pressure | t | T | Yield | Selectivity |
| (mol %) | (bar) | (h) | (° C.) | (%) | (%) |
| Co₂(CO)₈ | 50 | 18 | 80 | 18 | nd |
| (5) | | | | | |
| Co₂(CO)₈ | 15 | 18 | 80 | 23 | 47 |
| (5) | | | | | |
| Co₂(CO)₈ | 15 | 6 | 80 | 18 | 42 |
| (5) | | | | | |
| NaCo(CO)₄ | 15 | 6 | 80 | 21 | 64 |
| (5) | | | | | |
| Co₂(CO)₈ | 5 | 6 | 80 | 8 | 13 |
| (5) | constant | | | | |
| Co₂(CO)₈ | 15 | 6 | 110 | 5 | 9 |
| (5) | | | | | |

In this table, a constant pressure of 5 bar means that the pressure was maintained at 5 bar in carbon monoxide throughout the reaction.

The results presented in [Table 2] compare the effect of different carbon monoxide pressures on the yield of succinic anhydride.

Reducing the carbon monoxide pressure from 50 to 15 bar gives a slight increase in yield.

Interrupting the reaction after 6 h gave 78% of the yield obtained after 18 h. The experiments were then carried out over 6 hours for greater convenience.

At 15 bar carbon monoxide, the performance of Co₂(CO)₈ and NaCo(CO)₄ is similar.

Reducing the carbon monoxide pressure from 15 to 5 bar had a deleterious effect on the yield, as did increasing the temperature from 80 to 11000.

TABLE 3
Optimisation of the choice of the catalyst system (ligands
and additives), under carbon monoxide pressure alone
All the reactions were performed in toluene, with an acrylic
acid concentration of 1 mol/L, under CO pressure, with a
total pressure of 15 bar, T = 80° C.

| | | | | Succinic anhydride | |
|---|---|---|---|---|---|
| Catalyst | Ligand | Additive | t | Yield | Selectivity |
| (mol %) | (mol %) | (mol %) | (h) | (%) | (%) |
| Co₂(CO)₈ | | | 6 | 18 | 42 |
| (5) | | | | | |
| Co₂(CO)₈ | 1,2-bis(diphenyl- | | 6 | 7 | 50 |
| (5) | phosphino) | | | | |
| | ethane | | | | |
| | (5) | | | | |
| Co₂(CO)₈ | Pyridine | | 6 | 11 | 15 |
| (5) | (5) | | | | |
| Co₂(CO)₈ | 1,2-bis(diphenyl- | | 6 | 27 | 84 |
| (5) | phosphino | | | | |
| | methyl)benzene | | | | |
| | (5) | | | | |
| Co₂(CO)₈ | tris(pentafluro | | 6 | 30 | 57 |
| (5) | phenyl) | | | | |
| | phosphine | | | | |
| | (5) | | | | |
| Co₂(CO)₈ | | KI | 6 | 33 | 70 |
| (5) | | (5) | | | |
| Co₂(CO)₈ | Triphenyl | | 6 | 39 | 71 |
| (5) | phosphine | | | | |
| | (5) | | | | |
| Co₂(CO)₈ | s-IPr* (5) | | 6** | 42 | 67 |
| (5) | | | | | |
| Co₂(CO)₈ | | | 18 | 23 | 47 |
| (5) | | | | | |
| Co₂(CO)₈ | Tri-n- | | 18 | 21 | 28 |
| (5) | butylphosphine | | | | |
| | (5) | | | | |
| Co₂(CO)₈ | Triphenyl | | 18 | 66 | >99 |
| (5) | phosphine | | | | |
| | (5) | | | | |

*1,3-bis(2,6-diisopropylphenyl)-4,5-dihydro-1H-imidazol-3-ium.
**Experiment carried out at a temperature T of 90° C. (not 80° C.).

The results presented in [Table 3] show that compared to a system without ligands or additives:

The ligands that had a deleterious effect on the yield were: 1,2-bis(diphenyl phosphino)ethane and pyridine.

The ligand that had no significant effect on the yield was: tri-n-butylphosphine.

The additives and ligands that had a positive effect on the yield were: 1,2-bis(diphenylphosphinomethyl)benzene, tris(pentafluorophenyl)phosphine, potassium iodide, triphenylphosphine and 1,3-bis(2,6-diisopropylphenyl)-4,5-dihydro-1H-imidazol-3-ium (also referred to as s-IPr).

TABLE 4
Optimisation of the choice of catalyst system (source of
cobalt, ligands and additives) under pressure from
a mixture of carbon monoxide and dihydrogen
All reactions were performed in toluene, with an acrylic acid
concentration of 1 mol/L, under pressure of a CO/H₂ 95:5
mixture, with a total pressure of 16 bar, t = 6 h, T = 80° C.

| | | | Succinic anhydride | |
|---|---|---|---|---|
| Catalyst | Ligand | Additive | Yield | Selectivity |
| (mol %) | (mol %) | (mol %) | (%) | (%) |
| Co₂(CO)₈ | | | 16 | 41 |
| (5) | | | | |
| Co₂(CO)₈ | 1,2-bis | | 5 | 28 |
| (5) | (diphenylphosphino) | | | |
| | benzene | | | |
| | (5) | | | |
| Co₂(CO)₈ | Tris-[2- | | 12 | 24 |
| (5) | (diphenylphosphino) | | | |
| | ethyl]phosphine | | | |
| | (5) | | | |
| Co₂(CO)₈ | 1,3-bis | | 14 | 34 |
| (5) | (diphenylphosphino) | | | |
| | propane | | | |
| | (5) | | | |
| Co₂(CO)₈ | Xantphos | | 16 | 58 |
| (5) | (5) | | | |
| Co₂(CO)₈ | Tri-tert-butylphosphine | | 21 | 40 |
| (5) | (5) | | | |
| NaCo(CO)₄ | | | 22 | 56 |
| (5) | | | | |
| Co₂(CO)₈ | 1,2-bis | | 23 | 66 |
| (5) | (dimethylphosphino) | | | |
| | ethane | | | |
| | (5) | | | |
| Co₂(CO)₈ | | KI (5) | 24 | 46 |
| (5) | | | | |
| Co₂(CO)₈ | Trimethylphosphite | | 24 | 59 |
| (5) | (5) | | | |
| Co₂(CO)₈ | tris(pentafluorophenyl) | | 25 | 48 |
| (5) | phosphine | | | |
| | (5) | | | |
| Co₂(CO)₈ | Tributylphosphine | | 27 | 50 |
| (5) | (5) | | | |
| Co₂(CO)₈ | Diphenyl(pentafluoro- | | 38 | 63 |
| (5) | phenyl)phosphine | | | |
| | (5) | | | |
| Co₂(CO)₈ | Tri(o-tolyl)phosphine | | 41 | 59 |
| (5) | (5) | | | |
| Co₂(CO)₈ | Triphenylphosphine | | 41 | 76 |
| (5) | (5) | | | |
| Co₂(CO)₈ | 1,2-bis | | 49 | 82 |
| (5) | (dicyclohexylphosphino) | | | |
| | ethane | | | |
| | (5) | | | |
| Co₂(CO)₈ | 1,2-bis | KI (5) | 56 | 97 |
| (5) | (diphenylphosphino) | | | |
| | ethane | | | |
| | (5) | | | |
| Co₂(CO)₈ | 1,2-bis | | 63 | 81 |
| (5) | (diphenylphosphino) | | | |
| | ethane | | | |
| | (5) | | | |

The results presented in [Table 4] allow to compare the performance of different ligands and catalysts under pressure from a mixture of carbon monoxide and dihydrogen (95:5).

The ligands with a very positive effect on the yield of succinic anhydride are diphosphine ligands with an ethyl chain and phenyl and cyclohexyl substituents on the phosphorus (yield greater than 49%).

The ligands with a positive effect are monophosphine or monophosphite ligands, as well as the diphosphine ligand with an ethyl chain and methyl substituents on the phosphorus (yield between 21 and 41%)

The ligands with a negative effect or no effect were diphosphine ligands with a benzyl or propyl or xanthene chain, and triphosphine or tetraphosphine ligands (yields below 16%).

Under the conditions presented, NaCo(CO)₄ has a slightly positive effect on the yield compared with Co₂(CO)₈.

The potassium iodide has a positive effect on the yield; however, when combined with the ligand 1,2-bis(diphenylphosphino)ethane, it has a deleterious effect compared with a catalysis using the ligand alone.

TABLE 5
Optimisation of the acrylic acid concentration All reactions were
performed in toluene, under pressure of a CO/H₂ 95:5 mixture,
with a total pressure of 16 bar, t = 6 h, T = 80 ° C.

| | | Acrylic | Succinic anhydride | |
|---|---|---|---|---|
| Catalyst | Ligand | acid | Yield | Selectivity |
| (mol %) | (mol %) | (mol/L) | (%) | (%) |
| Co₂(CO)₈ | 1,2-bis(diphenylphosphino) | 1 | 63 | 81 |
| (5) | ethane | | | |
| | (5) | | | |
| Co₂(CO)₈ | 1,2-bis(diphenylphosphino) | 0.5 | 64 | >99 |
| (5) | ethane | | | |
| | (5) | | | |
| Co₂(CO)₈ | 1,2-bis(dicyclohexylphosphino) | 1 | 49 | 82 |
| (5) | ethane | | | |
| | (5) | | | |
| Co₂(CO)₈ | 1,2-bis | 0.5 | 89 | nd |
| (5) | (dicyclohexylphosphino)ethane | | | |
| | (5) | | | |

The results presented in [Table 5] allow us to compare the effect of the initial acrylic acid concentration on the succinic anhydride yield.

Decreasing the acrylic acid concentration had no effect in the case of the 1,2-bis(diphenylphosphino)ethane ligand system, but produced a very large increase in the yield in the case of the 1,2-bis(dicyclohexylphosphino)ethane ligand.

TABLE 6
Performance tests of different bidentate ferrocene ligands
compared to 1,2-bis(dicyclohexylphosphino)ethane ligand
All reactions were performed in toluene, with an acrylic acid
concentration of 0.5 mol/L, under pressure of a CO/H₂ 95:5
mixture, with a total pressure of 16 bar, t = 6 h, T = 90° C.

| | | Succinic anhydride | |
|---|---|---|---|
| Catalyst | Ligand | Yield | Selectivity |
| (mol %) | (mol %) | (%) | (%) |
| Co₂(CO)₈ | 1,2-bis(dicyclohexylphosphino)ethane | 96 | nd |
| (5) | (5) | | |
| Co₂(CO)₈ | 1,1′-bis(dicyclohexylphosphino) | 4 | 20 |
| (5) | ferrocene | | |
| | (5) | | |
| Co₂(CO)₈ | 1,1′-bis(diphenylphosphino)ferrocene | 26 | 46 |
| (5) | (5) | | |
| Co₂(CO)₈ | 1,1′-bis(diisopropylphosphino) | 37 | nd |
| (5) | ferrocene | | |
| | (5) | | |

The results presented in [Table 6] allow us to compare the performance of ferrocene ligands previously described in the literature as promoting the related hydroformylation reaction (Kluwer, A. M., Krafft, M. J., Hartenbach, I., de Bruin, B., & Kaim, W., Topics in Catalysis 2016, 59(19), 1787-1792 (doi:10.1007/s11244-016-0699-3)). The ferrocene ligands tested performed much less well than the 1,2-bis(dicyclohexylphosphino)ethane ligand, probably because of their insufficient solubility in toluene.

TABLE 7
Optimisation of the ligand and catalyst concentration
All reactions were performed in toluene, under pressure from a
CO/H₂ 95:5 mixture, with a total pressure of 16 bar, t = 6 h.

| | | | | Succinic | |
|---|---|---|---|---|---|
| | | Acrylic | | anhydride | |
| Catalyst | Ligand | acid | T | Yield | Selectivity |
| (mol %) | (mol %) | (mol/L) | (° C.) | (%) | (%) |
| Co₂(CO)₈ | Tri-n-butylphosphine | 1 | 80 | 27 | 50 |
| (5) | (5) | | | | |
| Co₂(CO)₈ | Tri-n-butylphosphine | 1 | 80 | 20 | 30 |
| (5) | (10) | | | | |
| Co₂(CO)₈ | 1,2-bis | 0.5 | 80 | 89 | nd |
| (5) | (dicyclohexylphosphino) | | | | |
| | ethane | | | | |
| | (5) | | | | |
| Co₂(CO)₈ | 1,2-bis | 0.5 | 80 | 80 | nd |
| (5) | (dicyclohexylphosphino) | | | | |
| | ethane | | | | |
| | (2,5) | | | | |
| Co₂(CO)₈ | 1,2-bis | 0.5 | 80 | 79 | >99 |
| (2, 5) | (dicyclohexylphosphino) | | | | |
| | ethane | | | | |
| | (2,5) | | | | |
| Co₂(CO)₈ | 1,2-bis | 0.5 | 90 | 56 | 90 |
| (2, 5) | (dicyclohexylphosphino) | | | | |
| | ethane | | | | |
| | (2,5) | | | | |
| Co₂(CO)₈ | 1,2-bis | 0.5 | 80 | 2 | 13 |
| (1) | (dicyclohexylphosphino) | | | | |
| | ethane | | | | |
| | (1) | | | | |
| Co₂(CO)₈ | 1,2-bis | 0.5 | 80 | traces | nd |
| (1) | (dicyclohexylphosphino) | | | | |
| | ethane | | | | |
| | (1) | | | | |

The results presented in [Table 7] compare the performance of catalyst systems containing different amounts of catalyst and ligand, relative to the amount of substrate introduced.

In the case of the tri-n-butylphosphine monophosphine ligand, the best yield is obtained when the ligand is introduced in an equimolar quantity relative to the carbonyl cobalt.

In the case of the ligand 1,2-bis(dicyclohexylphosphino)ethane, very good yields were obtained (>80%) for an amount greater than 2.5 mol % of Co₂(CO)₈ and ligand compared with acrylic acid.

TABLE 8
Catalysis with different gas compositions and different carbon
monoxide and dihydrogen pressures
All reactions were performed in toluene with 5 mol % Co₂(CO)₈ as catalyst.

| | Acrylic | | | | | Succinic anhydride | |
|---|---|---|---|---|---|---|---|
| Ligand | acid | | Pₜₒₜₐₗ | t | T | Yield | Selectivity |
| (mol %) | (mol/L) | Gaz | (bar) | (h) | (° C.) | (%) | (%) |
| | 1 | CO | 5 const | 6 | 80 | 8 | 13 |
| | 1 | CO | 15 | 6 | 80 | 18 | 42 |
| | 1 | CO/H₂ | 16 | 6 | 80 | 16 | 41 |
| | | 95:5 | | | | | |
| | 1 | CO | 15 | 18 | 80 | 23 | 47 |
| | 1 | CO | 50 | 18 | 80 | 18 | nd |
| Triphenyl- | 1 | CO | 15 | 6 | 80 | 39 | 71 |
| phosphine | | | | | | | |
| (5) | | | | | | | |
| Triphenyl- | 1 | CO/H₂ | 16 | 6 | 80 | 41 | 76 |
| phosphine | | 95:5 | | | | | |
| (5) | | | | | | | |
| 1,2-bis(diphenyl- | 1 | CO | 15 | 6 | 80 | 7 | 50 |
| phosphino) | | | | | | | |
| ethane | | | | | | | |
| (5) | | | | | | | |
| 1,2-bis(diphenyl- | 1 | CO/H₂ | 5 const | 6 | 80 | 15 | 39 |
| phosphino) | | 95:5 | | | | | |
| ethane | | | | | | | |
| (5) | | | | | | | |
| 1,2-bis(diphenyl- | 1 | CO/H₂ | 16 | 6 | 80 | 63 | 81 |
| phosphino) | | 95:5 | | | | | |
| ethane | | | | | | | |
| (5) | | | | | | | |
| 1,2-bis | 0.5 | CO | 15 | 6 | 90 | 19 | 40 |
| (dicyclohexyl- | | | | | | | |
| phosphino) | | | | | | | |
| ethane | | | | | | | |
| (5) | | | | | | | |
| 1,2-bis | 0.5 | CO/H₂ | 16 | 6 | 90 | 96 | nd |
| (dicyclohexyl- | | 95:5 | | | | | |
| phosphino) | | | | | | | |
| ethane | | | | | | | |
| (5) | | | | | | | |
| 1,2-bis | 0.5 | CO/H₂ | 50 | 6 | 90 | 48 | nd |
| (dicyclohexyl- | | 95:5 | | | | | |
| phosphino) | | | | | | | |
| ethane | | | | | | | |
| (5) | | | | | | | |
| | 1 | CO/H₂ | 30 | 6 | 80 | 10 | 10 |
| | | 1:1 | | | | | |

A constant pressure of 5 bar indicates that the pressure was maintained at 5 bar in carbon monoxide throughout the reaction.

The results presented in [Table 8] highlight the combined effects of gas composition, total pressure and ligand addition on the yield of succinic anhydride at the end of the reaction, when the reaction is performed in toluene.

In the absence of ligands, the best results are obtained at 15 bar total or partial carbon monoxide pressure. The presence of dihydrogen is therefore not harmful, but its addition is not necessary for better performance.

In the presence of monophosphine ligands, the reaction can be performed either under carbon monoxide alone (15 bar) or under the pressure of a mixture of carbon monoxide and dihydrogen (95:5, 16 bar). The presence of dihydrogen is therefore not harmful, but its addition is not necessary for better performance.

In the presence of diphosphine ligands, the reaction has a very low yield under carbon monoxide alone (15 bar), and a very high yield under pressure of a mixture of carbon monoxide and dihydrogen (95:5, 16 bar). The presence of dihydrogen in the gas phase is therefore necessary for the catalytic system to be performant.

The reaction under 15 bar of CO and 15 bar of H₂ (6 h, 80° C.) gives only a low yield (10%), so the proportion of dihydrogen in the CO/H₂ mixture should be limited.

TABLE 9
Optimisation of the reaction time
All reactions were performed in toluene with 5 mol % Co₂(CO)₈ as catalyst.

| | Acrylic | | | | | Succinic anhydride | |
|---|---|---|---|---|---|---|---|
| Ligand | acid | | Pₜₒₜₐₗ | t | T | Yield | Selectivity |
| (mol %) | (mol/L) | Gaz | (bar) | (h) | (° C.) | (%) | (%) |
| | 1 | CO | 15 | 6 | 80 | 18 | 42 |
| | 1 | CO | 15 | 18 | 80 | 23 | 47 |
| | 1 | CO/H₂ | 16 | 3 | 80 | 12 | 34 |
| | | 95:5 | | | | | |
| | 1 | CO/H₂ | 16 | 6 | 80 | 16 | 41 |
| | | 95:5 | | | | | |
| | 1 | CO/H₂ | 16 | 18 | 80 | 17 | 44 |
| | | 95:5 | | | | | |
| 1,2- | 1 | CO/H₂ | 16 | 3 | 80 | 51 | 82 |
| bis(diphenyl- | | 95:5 | | | | | |
| phosphino) | | | | | | | |
| ethane | | | | | | | |
| (5) | | | | | | | |
| 1,2- | 1 | CO/H₂ | 16 | 6 | 80 | 63 | 81 |
| bis(diphenyl- | | 95:5 | | | | | |
| phosphino) | | | | | | | |
| ethane | | | | | | | |
| (5) | | | | | | | |
| 1,2- | 1 | CO/H₂ | 16 | 18 | 80 | 78 | 92 |
| bis(diphenyl- | | 95:5 | | | | | |
| phosphino) | | | | | | | |
| ethane | | | | | | | |
| (5) | | | | | | | |
| 1,2- | 1 | CO/H₂ | 16 | 69 | 80 | 88 | >99 |
| bis(diphenyl- | | 95:5 | | | | | |
| phosphino) | | | | | | | |
| ethane | | | | | | | |
| (5) | | | | | | | |
| 1,2- | 0.5 | CO/H₂ | 16 | 1 | 150 | 20 | 31 |
| bis(dicyclohexyl- | | 95:5 | | | | | |
| phosphino) | | | | | | | |
| ethane | | | | | | | |
| (5) | | | | | | | |
| 1,2- | 0.5 | CO/H₂ | 16 | 3 | 150 | 28 | nd |
| bis (dicyclohexyl- | | 95:5 | | | | | |
| phosphino) | | | | | | | |
| ethane | | | | | | | |
| (5) | | | | | | | |
| 1,2-bis | 0.5 | CO/H₂ | 16 | 2 | 120 | 55 | 55 |
| (dicyclohexyl- | | 95:5 | | | | | |
| phosphino) | | | | | | | |
| ethane | | | | | | | |
| (5) | | | | | | | |
| 1,2-bis | 0.5 | CO/H₂ | 16 | 3 | 110 | 96 | 96 |
| (dicyclohexyl- | | 95:5 | | | | | |
| phosphino) | | | | | | | |
| ethane | | | | | | | |
| (5) | | | | | | | |
| 1,2-bis | 0.5 | CO/H₂ | 16 | 3 | 90 | 55 | nd |
| (dicyclohexyl- | | 95:5 | | | | | |
| phosphino) | | | | | | | |
| ethane | | | | | | | |
| (5) | | | | | | | |
| 1,2-bis | 0.5 | CO/H₂ | 16 | 6 | 90 | 96 | nd |
| (dicyclohexyl- | | 95:5 | | | | | |
| phosphino) | | | | | | | |
| ethane | | | | | | | |
| (5) | | | | | | | |
| 1,2-bis | 0.5 | CO/H₂ | 16 | 6 | 80 | 89 | nd |
| (dicyclohexyl- | | 95:5 | | | | | |
| phosphino) | | | | | | | |
| ethane | | | | | | | |
| (5) | | | | | | | |
| 1,2-bis | 0.5 | CO/H₂ | 16 | 18 | 60 | 14 | 39 |
| (dicyclohexyl- | | 95:5 | | | | | |
| phosphino) | | | | | | | |
| ethane | | | | | | | |
| (5) | | | | | | | |

The results presented in [Table 9] give an indication of the reaction kinetics under different conditions; some results are highlighted in FIG. 5, which shows the kinetic curves under different conditions.

The diphosphine ligands with ethyl chains and cyclohexyl or phenyl substituents are particularly effective. The kinetic curves suggest that the yield is limited to less than 25% when using Co₂(CO)₈ alone.

When the ligand 1,2-bis(dicyclohexylphosphino)ethane is used, it is equivalent to carrying out the reaction for 6 h at 90° C. or to carrying out for 3 h at 11000. However, reducing the temperature to 60° C. leads to a very significant drop in yield, despite a reaction time of 18 h. Raising the temperature above 120° C. is detrimental to the yield.

TABLE 10
Optimising the choice of the solvent
All the reactions were performed with 5 mol % Co₂(CO)₈ as catalyst.

| | | Acrylic | | | | | Succinic anhydride | |
|---|---|---|---|---|---|---|---|---|
| Ligand | | acid | | | t | T | Yield | Selectivity |
| (mol %) | Solvent | (mol/L) | Gaz | Pₜₒₜₐₗ | (h) | (° C.) | (%) | (%) |
| | Nitromethane | 0.5 | CO | 15 | 6 | 90 | 0 | 0 |
| | Pentane | 0.5 | CO | 15 | 6 | 90 | 0 | 0 |
| | Hexane | 0.5 | CO | 15 | 6 | 90 | 0 | 0 |
| | Pyridine | 0.5 | CO | 15 | 6 | 90 | 0 | 0 |
| | Pyridine | 0.5 | CO/H₂ | 16 | 6 | 90 | 0 | 0 |
| | | | 95:5 | | | | | |
| | Dimethyl- | 0.5 | CO | 16 | 6 | 90 | 0 | 0 |
| | formamide | | | | | | | |
| | Dimethyl- | 0.5 | CO/H₂ | 16 | 6 | 90 | O | 0 |
| | formamide | | 95:5 | | | | | |
| 1,2-bis | Dimethyl- | 0.5 | CO/H₂ | 16 | 6 | 90 | 0 | 0 |
| (dicyclohexyl | formamide | | 95:5 | | | | | |
| phosphino) | | | | | | | | |
| ethane | | | | | | | | |
| (5) | | | | | | | | |
| | Diethyl ether | 0.5 | CO | 15 | 6 | 90 | 6 | 16 |
| | Toluene | 0.5 | CO | 15 | 6 | 80 | 17 | 45 |
| 1,2-bis | Toluene | 0.5 | CO/H₂ | 16 | 6 | 90 | 96 | nd |
| (dicyclohexyl | | | 95:5 | | | | | |
| phosphino) | | | | | | | | |
| ethane | | | | | | | | |
| (5) | | | | | | | | |
| | Dichloro | 0.5 | CO | 15 | 6 | 90 | 18 | 29 |
| | methane | | | | | | | |
| | Tetrahydro- | 0.5 | CO | 15 | 6 | 90 | 24 | 40 |
| | furan | | | | | | | |
| | Dimethoxy | 0.5 | CC | 15 | 6 | 90 | 29 | 85 |
| | ethane | | | | | | | |
| | Dimethoxy | 0.5 | CO/H₂ | 16 | 6 | 90 | 46 | 49 |
| | ethane | | 95:5 | | | | | |
| 1,2-bis | Dimethoxy- | 0.5 | CO/H₂ | 16 | 6 | 80 | 52 | 96 |
| (dicyclohexyl | ethane | | 95:5 | | | | | |
| phosphino) | | | | | | | | |
| ethane | | | | | | | | |
| (5) | | | | | | | | |
| | Acetate | 0.5 | CO | 15 | 6 | 90 | 41 | 55 |
| | ethyl | | | | | | | |
| | Dioxane | 0.5 | CC | 15 | 6 | 90 | 27 | nd |
| | Dioxane | 0.5 | CO/H₂ | 16 | 6 | 90 | 41 | nd |
| | | | 95:5 | | | | | |
| 1,2-bis | Dioxane | 0.5 | CO/H₂ | 16 | 6 | 80 | 45 | nd |
| (dicyclohexyl | | | 95:5 | | | | | |
| phosphino) | | | | | | | | |
| ethane | | | | | | | | |
| (5) | | | | | | | | |
| | Propylene | 0.5 | CO | 15 | 6 | 90 | 44 | 53 |
| | carbonate | | | | | | | |
| | Acetone | 0.5 | CO | 15 | 6 | 90 | 62 | 62 |
| | Acetonitrile | 0.5 | CO | 15 | 6 | 90 | 61 | 76 |
| | Acetonitrile | 0.5 | CO/H₂ | 16 | 6 | 90 | 68 | 80 |
| | | | 95:5 | | | | | |
| 1,2-bis | Acetonitrile | 0.5 | CO/H₂ | 16 | 6 | 90 | 3 | 23 |
| (dicyclohexyl | | | 95:5 | | | | | |
| phosphino) | | | | | | | | |
| ethane (5) | | | | | | | | |
| 1,2-bis | Acetonitrile | 1 | CO/H₂ | 16 | 6 | 80 | 10 | 33 |
| (diphenyl- | | | 95:5 | | | | | |
| phosphino) | | | | | | | | |
| ethane | | | | | | | | |
| (5) | | | | | | | | |

The results presented in [Table 10] allow a comparison of the effects due to the solvents, under the conditions indicated.

The pyridine, dimethylformamide, nitromethane, pentane and hexane do not appear to be suitable solvents for the reaction (no succinic anhydride observed at the end of the reaction).

In the absence of ligands and without the addition of H₂, the solvents ranging from the least to the most efficient are: diethyl ether, toluene, dichloromethane, tetrahydrofuran, 1,4-dioxane, dimethoxyethane, ethyl acetate, propylene carbonate, acetonitrile and acetone.

In the presence of the ligand 1,2-bis(dicyclohexylphosphino)ethane and with the addition of H₂, the solvents with the lowest to highest performance were: acetonitrile, 1,4-dioxane, dimethoxyethane and toluene.

The solvents for which the addition of H₂ and/or ligands has little or no effect are dimethylformamide, pyridine and dioxane.

The solvents for which the addition of H₂ and/or ligands has a slight effect are dimethoxyethane.

The solvents for which the addition of H₂ and/or ligands has a major effect are acetonitrile, toluene and 1,4-dioxane.

TABLE 11
Study of the performance of the catalytic system in acetonitrile
All reactions were performed in acetonitrile with 5 mol % Co₂(CO)₈ as catalyst.

| | Acrylic | | | | | Succinic anhydride | |
|---|---|---|---|---|---|---|---|
| Ligand | acid | | | t | T | Yield | Selectivity |
| (mol %) | (mol/L) | Gaz | Ptotal | (h) | (° C.) | (%) | (%) |
| | 0.5 | CO | 15 | 18 | 30 | 0 | 0 |
| | 0.5 | CC | 15 | 6 | 70 | 44 | 56 |
| | 0.5 | CO | 15 | 3 | 90 | 37 | 46 |
| | 0.5 | CO | 15 | 6 | 90 | 61 | 76 |
| | 0.5 | CO | 15 | 18 | 90 | 61 | 73 |
| | 0.5 | CO | 15 | 3 | 110 | 32 | 53 |
| | 0.5 | CO | 15 | 3 | 130 | 17 | nd |
| | 0.5 | CO | 15 | 3 | 150 | 9 | 23 |
| | 0.5 | CO | 50 | 6 | 90 | 72 | 78 |
| | 0.5 | CO | 100 | 6 | 90 | 20 | 41 |
| | 0.5 | CO/H₂ | 16 | 6 | 90 | 68 | 80 |
| | | 95:5 | | | | | |
| 1,2- | 0.5 | CO | 15 | 6 | 90 | 22 | 48 |
| bis(dicyclohexyl- | | | | | | | |
| phosphino) | | | | | | | |
| ethane (5) | | | | | | | |
| 1,2-bis | 0.5 | CO/H₂ | 16 | 6 | 90 | 3 | 23 |
| (dicyclohexyl | | 95:5 | | | | | |
| phosphino) | | | | | | | |
| ethane (5) | | | | | | | |
| 1,2-bis(diphenyl- | 1 | CO/H₂ | 16 | 6 | 80 | 10 | 33 |
| phosphino) | | 95:5 | | | | | |
| ethane | | | | | | | |
| (5) | | | | | | | |

The results presented in [Table 11] give more information on the use of acetonitrile as a solvent. Compared to the other solvents tested, acetonitrile has a particular behaviour: it gives a good yield in the absence of ligand (68%), but only traces of product are observed in the presence of ligands (3% or 10%). Adding ligands to acetonitrile would therefore seem to be an unfavourable option.

The absence of dihydrogen in the gas phase only leads to a very slight drop in yield.

The yield of succinic anhydride obtained after 18 h of reaction at 90° C. under 15 bar of CO was the same as after 6 h of reaction: the yield therefore appears to be limited to around 60% when acetonitrile is used as the solvent.

Increasing the carbon monoxide pressure from 15 to 50 bar allows to improve the yield to 72%, but increasing it further to 100 bar is highly detrimental to the yield.

The optimum temperature is 90° C.

TABLE 12
Application to acrylic acid derivatives
All the reactions were performed in acetonitrile, with
an unsaturated acid concentration of 0.5 mol/L, under
CO pressure, with a total pressure of 15 bar, T = 110° C.

| | | | Succinic anhydride | |
|---|---|---|---|---|
| Unsaturated acid | Catalyst | t | Yield | Selectivity |
| (mol %) | (mol %) | (h) | (%) | (%) |
| Crotonic acid | Co₂(CO)₈ | 20 | 22 | 41 |
| | (5) | | | |
| Crotonic acid | Co₂(CO)₈ | 6 | 45% | 76% |
| | (10) | | | |
| Crotonic acid | Co₂(CO)₈ | 18 | 56% | nd |
| | (10) | | | |
| Crotonic acid | Co₂(CO)₈ | 100 | 68% | 68% |
| | (10) | | | |
| Methacrylic acid | Co₂(CO)₈ | 100 | 24% | 25% |
| | (10) | | | |
| 2-Pentenoic acid | Co₂(CO)₈ | 18 | 21% | nd |
| | (10) | | | |

The results presented in Table 12 show that the reaction can be applied to acrylic acid derivatives. Under the conditions indicated, the presence of R₁, R₂ or R₃ groups other than hydrogen leads to a reduction in catalytic activity. This drop is more marked if the group is in position R₂ than in position R₁, and is accompanied by a drop in selectivity. When the group is in position R₁,the catalytic activity decreases with the steric hindrance of the group.

TABLE 13
Use of other metal catalysts
The reaction was performed in dimethoxyethane,
under pressure of a CO/H₂ 95:5 mixture, with a
total pressure of 16 bar, t = 65 h, T = 110 ° C.

| | | Succinic anhydride | |
|---|---|---|---|
| Catalyst | Ligand | Yield | Selectivity |
| (mol %) | (mol %) | (%) | (%) |
| Palladium (II) | 1,3-bis(diphenylphosphino) | 24% | nd |
| trifluoroacetate | propane | | |
| (5) | (5) | | |

The results presented in [Table 13] show that palladium can be used as a catalyst in the reaction.

1. A method for preparing a cyclic anhydride of formula (I) 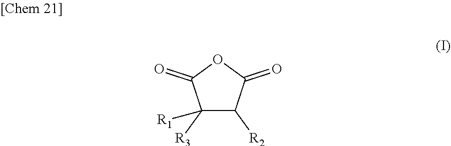
wherein
R₁, R₂ and R₃, which may be identical or different, represent a hydrogen atom, an alkyl radical containing 1 to 25 carbon atoms, a cycloalkyl radical containing 3 to 12 carbon atoms, an aryl radical containing 6 to 20 carbon atoms, a hydroxyl group, a nitrile group, an amine group, an amide group, a carbonyl group, a carboxylate group, a nitro (NO₂) group, an alkoxy group, an aryloxy group or a halogen atom, said alkyl, cycloalkyl and aryl radicals being optionally substituted;
characterised in that an acid of formula (II) 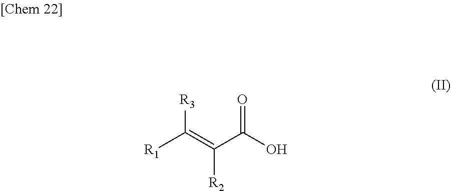
wherein R₁, R₂ and R₃ are as defined above;
is reacted with carbon monoxide (CO) or a mixture of carbon monoxide and hydrogen (CO/H₂), in the presence of a metal catalyst of formula (III)
[Chem 23]
LₐM_{b}X_{c}(CO)_{d} (III)
wherein
L represents Na⁺, K⁺, Li⁺, Cs⁺, Mg²⁺, Ca²⁺, NH₄⁺, [((C₆H₅)₃P)₂N]⁺; a ligand chosen from:
the phosphines of formula PR₄R₅R₆, in which R₄, R₅ and R₆, which may be identical or different, represent a hydrogen atom, an alkyl radical containing 1 to 12 carbon atoms, an aryl radical containing from 6 to 20 carbon atoms, an alkoxy group, an aryloxy group or a ferrocenyl group, said alkyl, aryl and ferrocenyl radicals being optionally substituted;
the polyphosphines which mean a phosphine as defined above, in which at least one of the substituents R₄, R₅ and R₆ is substituted by one or several phosphinyl groups of formula —PR₁₁R₁₂, in which R₁₁ and R₁₂, which may be identical or different, represent an alkyl radical containing 1 to 12 carbon atoms, an aryl radical containing from 6 to 20 carbon atoms, an alkoxy group, an aryloxy group, said alkyl and aryl radicals being optionally substituted;
the amines of formula NR₇R₈R₉ in which R₇, R₈ and R₉, which may be identical or different, represent a hydrogen atom, an alkyl radical containing 1 to 12 carbon atoms or an aryl radical containing from 6 to 20 carbon atoms, said alkyl and aryl radicals being optionally substituted;
the amides of formula R₇CO—NR₈R₉ in which R₇, R₈ and R₉, which may be identical or different, represent a hydrogen atom, an alkyl radical containing 1 to 12 carbon atoms or an aryl radical containing from 6 to 20 carbon atoms, said alkyl and aryl radicals being optionally substituted;
the N-heterocyclic carbenes optionally derived from an imidazolium salt, chosen from the 1,3-bis(2,6-diisopropylphenyl)-1H-imidazol-3-ium salts (also referred to as IPr), 1,3-bis(2,6-diisopropylphenyl)-4,5-dihydro-1H-imidazol-3-ium (also referred to as s-IPr), 1,3-bis(2,4,6-trimethylphenyl)-1H-imidazol-3-ium (also referred to as IMes), 1,3-bis(2,4,6-trimethylphenyl)-4,5-dihydro-1H-imidazol-3-ium (also referred to as s-IMes), 4,5-dichloro-1,3-bis(2,6-diisopropylphenyl)-1H-imidazol-3-ium (also referred to as Cl₂—IPr), 1,3-di-tert-butyl-1H-imidazol-3-ium (also referred to as ItBu), and 1,3-di-tert-butyl-4,5-dihydro-1H-imidazol-3-ium (also referred to as s-ItBu), said salts being in the form of chloride or tetraphenylborate salts;
cyclopentadienyl and its derivatives selected from 1,2,3,4,5pentakis(methyl)cyclopentadienyl, 1,2,3,4,5pentakis(1methylethyl)cyclopentadienyl and 1,2,3,4,5pentakis(1dimethylethyl)cyclopentadienyl;
the nitriles of formula NCR₁₀ in which R₁₀ represents an alkyl radical containing 1 to 12 carbon atoms or an aryl radical containing 6 to 20 carbon atoms, said alkyl and aryl radicals being optionally substituted;
the pyridine and its derivatives of formula (IV) 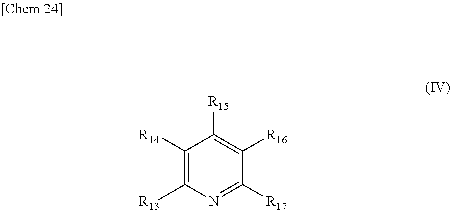
in which R₁₃, R₁₄, R₁₅, R₁₆ and R₁₇, which may be identical or different, represent a hydrogen atom, an alkyl radical containing 1 to 12 carbon atoms or an aryl radical containing 6 to 20 carbon atoms, said alkyl and aryl radicals being optionally substituted;
the bipyridines of formula (V) 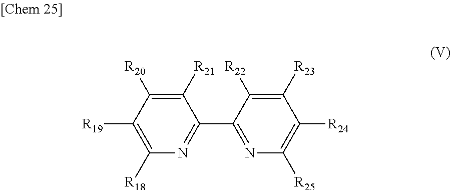
in which R₁₈, R₁₉, R₂₀, R₂₁, R₂₂, R₂₃, R₂₄ and R₂₅, which may be identical or different, represent a hydrogen atom, an alkyl radical containing 1 to 12 carbon atoms or an aryl radical containing 6 to 20 carbon atoms, said alkyl and aryl radicals being optionally substituted;
M represents Co, Fe, Mn, Rh, Ru, Pd; M representing in particular Co or Pd;
X represents a halide selected from F⁻, Cl⁻, Br⁻, I⁻; a trifluoromethylsulphonate (or triflate or CF₃SO₃);
an acetylacetonate (or acac or C₅H₇O₂⁻); an acetate (or CH₃COO⁻ or AcO⁻); a trifluoroacetate (or CF₃COO⁻ or TFAcO⁻);
a=0 to 12;
b=0 to 3;
c=0 to 12;
d=0 to 12;
in one or a mixture of at least two solvents chosen from:
the aromatic hydrocarbons chosen from benzene and toluene,
the nitriles chosen from acetonitrile, propionitrile and benzonitrile,
the linear or cyclic ethers chosen from diethyl ether, methyl vinyl ether, 1,1-diethoxyethane, dimethoxyethane, THF, methyltetrahydrofuran, tetrahydropyran and dioxane,
the esters such as ethyl acetate,
the carbonate esters such as propylene carbonate,
the ketones chosen from acetone and butanone,
the alkyl halides chosen from chloroform and methylene chloride,
the aryl halides chosen from chlorobenzene and dichlorobenzene;
at a temperature of 110° C. or less.

2. The method according to claim 1, wherein R₁, R₂ and R₃, which may be identical or different, represent a hydrogen atom, an alkyl radical containing 1 to 25 carbon atoms, an aryl radical containing 6 to 20 carbon atoms, a hydroxyl group, an alkoxy group or an aryloxy group, said alkyl and aryl radicals being optionally substituted.

3. The method according to one of claims 1, wherein
the metal catalyst is of formula (III)
[Chem 26]
LₐM_{b}X_{c}(CO)_{d} (III)
wherein
L represents Na⁺, K⁺, Li⁺, Cs⁺, Mg²⁺, Ca²⁺, NH₄⁺, [((C₆H₅)₃P)₂N]⁺; a ligand chosen from:
the phosphines of formula PR₄R₅R₆, in which R₄, R₅ and R₆, which may be identical or different, represent a hydrogen atom, an alkyl radical containing 1 to 12 carbon atoms, an aryl radical containing from 6 to 20 carbon atoms, an alkoxy group, an aryloxy group or a ferrocenyl group, said alkyl, aryl and ferrocenyl radicals being optionally substituted;
the polyphosphines which mean a phosphine as defined above, in which at least one of the substituents R₄, R₅ and R₆ is substituted by one or several phosphinyl groups of formula —PR₁₁R₁₂, in which R₁₁ and R₁₂, which may be identical or different, represent an alkyl radical containing 1 to 12 carbon atoms, an aryl radical containing from 6 to 20 carbon atoms, an alkoxy group, an aryloxy group, said alkyl and aryl radicals being optionally substituted;
amines of formula NR₇R₈R₉ in which R₇, R₈ and R₉, which may be identical or different, represent an alkyl radical containing 1 to 12 carbon atoms, an aryl radical containing from 6 to 20 carbon atoms, said alkyl and aryl radicals being optionally substituted;
the N-heterocyclic carbenes optionally derived from an imidazolium salt, chosen from the 1,3-bis(2,6-diisopropylphenyl)-1H-imidazol-3-ium salts (also referred to as IPr), 1,3-bis(2,6-diisopropylphenyl)-4,5-dihydro-1H-imidazol-3-ium (also referred to as s-IPr), 1,3-bis(2,4,6-trimethylphenyl)-1H-imidazol-3-ium (also referred to as IMes), 1,3-bis(2,4,6-trimethylphenyl)-4,5-dihydro-1H-imidazol-3-ium (also referred to as s-IMes), 4,5-dichloro-1,3-bis(2,6-diisopropylphenyl)-1H-imidazol-3-ium (also referred to as Cl₂—IPr), 1,3-di-tert-butyl-1H-imidazol-3-ium (also referred to as ItBu), and 1,3-di-tert-butyl-4,5-dihydro-1H-imidazol-3-ium (also referred to as s-ItBu), said salts being in the form of chloride or tetraphenylborate salts;
nitriles of formula NCR₁₀ in which R₁₀ represents an alkyl radical containing 1 to 12 carbon atoms or an aryl radical containing 6 to 20 carbon atoms, said alkyl and aryl radicals being optionally substituted;
the pyridine and its derivatives of formula (IV) 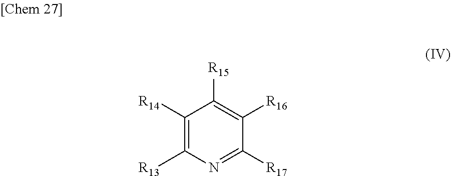
in which R₁₃, R₁₄, R₁₅, R₁₆ and R₁₇, which may be identical or different, represent a hydrogen atom, an alkyl radical containing 1 to 12 carbon atoms or an aryl radical containing 6 to 20 carbon atoms, said alkyl and aryl radicals being optionally substituted;
the bipyridines of formula (V) 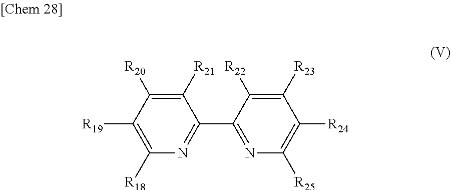
in which R₁₈, R₁₉, R₂₀, R₂₁, R₂₂, R₂₃, R₂₄ and R₂₅, which may be identical or different, represent a hydrogen atom, an alkyl radical containing 1 to 12 carbon atoms or an aryl radical containing 6 to 20 carbon atoms, said alkyl and aryl radicals being optionally substituted;
M represents Co, Fe, Rh, Pd; M representing in particular Co or Pd;
X represents a halide selected from F⁻, Cl⁻, Br⁻, I⁻; a trifluoromethylsulphonate (or triflate or CF₃SO₃);
an acetylacetonate (or acac or C₅H₇O₂⁻); an acetate (or CH₃COO⁻ or AcO⁻); a trifluoroacetate (or CF₃COO⁻ or TFAcO⁻);
a=0 to 12;
b=0 to 3;
c=0 to 12;
d=0 to 12.

4. The method according to claim 1, wherein the metal catalyst is of formula (III),
[Chem 29]
LₐM_{b}X_{c}(CO)_{d} (III)
wherein
L represents Na⁺, K⁺, NH₄⁺, [((C₆H₅)₃P)₂N]⁺; a ligand chosen from
the phosphines of formula PR₄R₅R₆, in which R₄, R₅ and R₆, which may be identical or different, represent a hydrogen atom, an alkyl radical containing 1 to 12 carbon atoms, an aryl radical containing from 6 to 20 carbon atoms, an alkoxy group, an aryloxy group or a ferrocenyl group, said alkyl, aryl and ferrocenyl radicals being optionally substituted;
the polyphosphines which mean a phosphine as defined above, in which at least one of the substituents R₄, R₅ and R₆ is substituted by one or several phosphinyl groups of formula —PR₁₁R₁₂, in which R₁₁ and R₁₂, which may be identical or different, represent an alkyl radical containing 1 to 12 carbon atoms, an aryl radical containing from 6 to 20 carbon atoms, an alkoxy group, an aryloxy group, said alkyl and aryl radicals being optionally substituted;
the N-heterocyclic carbenes optionally derived from an imidazolium salt, chosen from the 1,3-bis(2,6-diisopropylphenyl)-1H-imidazol-3-ium salts (also referred to as IPr), 1,3-bis(2,6-diisopropylphenyl)-4,5-dihydro-1H-imidazol-3-ium (also referred to as s-IPr), 1,3-bis(2,4,6-trimethylphenyl)-1H-imidazol-3-ium (also referred to as IMes), 1,3-bis(2,4,6-trimethylphenyl)-4,5-dihydro-1H-imidazol-3-ium (also referred to as s-IMes), 4,5-dichloro-1,3-bis(2,6-diisopropylphenyl)-1H-imidazol-3-ium (also referred to as Cl₂—IPr), 1,3-di-tert-butyl-1H-imidazol-3-ium (also referred to as ItBu), and 1,3-di-tert-butyl-4,5-dihydro-1H-imidazol-3-ium (also referred to as s-ItBu), said salts being in the form of chloride or tetraphenylborate salts;
the pyridine and its derivatives of formula (IV) 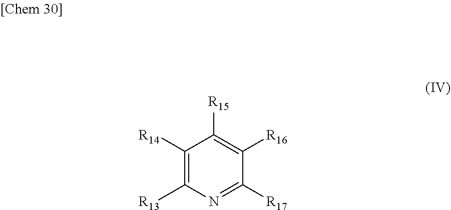
in which R₁₃, R₁₄, R₁₅, R₁₆ and R₁₇, which may be identical or different, represent a hydrogen atom, an alkyl radical containing 1 to 12 carbon atoms or an aryl radical containing 6 to 20 carbon atoms, said alkyl and aryl radicals being optionally substituted;
the bipyridines of formula (V) 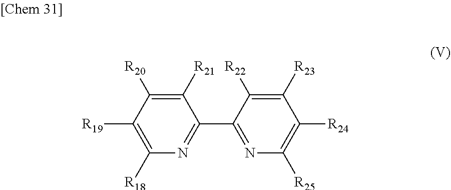
in which R₁₈, R₁₉, R₂₀, R₂₁, R₂₂, R₂₃, R₂₄ and R₂₅, which may be identical or different, represent a hydrogen atom, an alkyl radical containing 1 to 12 carbon atoms or an aryl radical containing 6 to 20 carbon atoms, said alkyl and aryl radicals being optionally substituted;
M represents Co or Pd; M representing Co in particular;
X represents a halide selected from Cl⁻, Br⁻; acetylacetonate (or acac or C₅H₇O₂⁻); acetate (or CH₃COO⁻ or AcO⁻); trifluoroacetate (or CF₃COO⁻ or TFAcO⁻);
a=0 to 8;
b=1 to 2;
c=0 to 8;
d=0 to 8.

5. The method according to claim 1, wherein it takes place in the presence
of an additive of formula ZY in which
Y represents an anion selected from F⁻, Cl⁻, Br⁻, I⁻; and
Z represents a cation chosen from Na⁺, K⁺, Li⁺, Cs⁺, Mg²⁺, Ca²⁺, NH₄⁺;
and/or
of a ligand chosen from:
the phosphines of formula PR₄R₅R₆, in which R₄, R₅ and R₆, which may be identical or different, represent a hydrogen atom, an alkyl radical containing 1 to 12 carbon atoms, an aryl radical containing from 6 to 20 carbon atoms, an alkoxy group, an aryloxy group or a ferrocenyl group, said alkyl, aryl and ferrocenyl radicals being optionally substituted;
the polyphosphines which mean a phosphine as defined above, in which at least one of the substituents R₄, R₅ and R₆ is substituted by one or several phosphinyl groups of formula —PR₁₁R₁₂, in which R₁ and R₁₂, which may be identical or different, represent an alkyl radical containing 1 to 12 carbon atoms, an aryl radical containing from 6 to 20 carbon atoms, an alkoxy group, an aryloxy group, said alkyl and aryl radicals being optionally substituted;
the amines of formula NR₇R₈R₉ in which R₇, R₈ and R₉, which may be identical or different, represent a hydrogen atom, an alkyl radical containing 1 to 12 carbon atoms or an aryl radical containing from 6 to 20 carbon atoms, said alkyl and aryl radicals being optionally substituted;
the amides of formula R₇CO—NR₈R₉ in which R₇, R₈ and R₉, which may be identical or different, represent a hydrogen atom, an alkyl radical containing 1 to 12 carbon atoms or an aryl radical containing from 6 to 20 carbon atoms, said alkyl and aryl radicals being optionally substituted;
the N-heterocyclic carbenes optionally derived from an imidazolium salt, chosen from the 1,3-bis(2,6-diisopropylphenyl)-1H-imidazol-3-ium salts (also referred to as IPr), 1,3-bis(2,6-diisopropylphenyl)-4,5-dihydro-1H-imidazol-3-ium (also referred to as s-IPr), 1,3-bis(2,4,6-trimethylphenyl)-1H-imidazol-3-ium (also referred to as IMes), 1,3-bis(2,4,6-trimethylphenyl)-4,5-dihydro-1H-imidazol-3-ium (also referred to as s-IMes), 4,5-dichloro-1,3-bis(2,6-diisopropylphenyl)-1H-imidazol-3-ium (also referred to as Cl₂—IPr), 1,3-di-tert-butyl-1H-imidazol-3-ium (also referred to as ItBu), and 1,3-di-tert-butyl-4,5-dihydro-1H-imidazol-3-ium (also referred to as s-ItBu), said salts being in the form of chloride or tetraphenylborate salts;
cyclopentadienyl and its derivatives selected from 1,2,3,4,5-pentakis(methyl)cyclopentadienyl, 1,2,3,4,5-pentakis(1-methylethyl)cyclopentadienyl and 1,2,3,4,5-pentakis(1-dimethylethyl)cyclopentadienyl;
the nitriles of formula NCR₁₀ in which R₁₀ represents an alkyl radical containing 1 to 12 carbon atoms or an aryl radical containing 6 to 20 carbon atoms, said alkyl and aryl radicals being optionally substituted;
the pyridine and its derivatives of formula (IV) 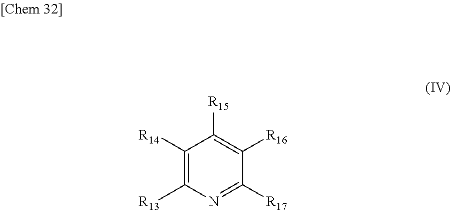
in which R₁₃, R₁₄, R₁₅, R₁₆ and R₁₇, which may be identical or different, represent a hydrogen atom, an alkyl radical containing 1 to 12 carbon atoms or an aryl radical containing 6 to 20 carbon atoms, said alkyl and aryl radicals being optionally substituted;
the bipyridines of formula (V) 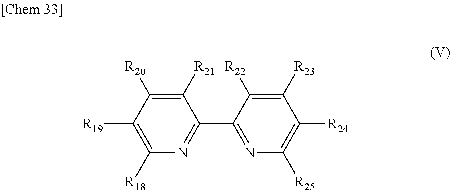
in which R₁₈, R₁₉, R₂₀, R₂₁, R₂₂, R₂₃, R₂₄ and R₂₅, which may be identical or different, represent a hydrogen atom, an alkyl radical containing 1 to 12 carbon atoms or an aryl radical containing 6 to 20 carbon atoms, said alkyl and aryl radicals being optionally substituted.

6. The method according to, wherein it takes place under a total pressure of carbon monoxide (CO) or of a mixture of carbon monoxide and dihydrogen (CO/H₂) of 5 to 60 bar.

7. The method according to claim 6, wherein the mixture of carbon monoxide and dihydrogen (CO/H₂) the proportion of dihydrogen is from 1 to 50%.

8. The method according to claim 1, wherein it occurs at a temperature of between 4° and 110° C.

9. The method according to claim 1, wherein the amount of catalyst of formula (III) is from 0.1 to 10 mol %, relative to the amount of acid of formula (II).

10. The method according to claim 1, wherein the concentration of the acid of formula (II) is between 0.01 and 5 M.

11. The method according to claim 1, wherein when it takes place in the presence of an additive, the quantity of additive is from 0.5 to 15 molar equivalents relative to the catalyst of formula (III), preferably from 1 to 10 equivalents, more preferably from 1 to 5 equivalents;
when it takes place in the presence of ligand, the amount of ligand is:
by 1 molar equivalent relative to the catalyst of formula (III) when the ligand is a polyphosphine;
from 1 to 2 molar equivalents relative to the catalyst of formula (III) when the ligand is a phosphine, preferably 1 molar equivalent;
from 0.5 to 12 molar equivalents relative to the catalyst of formula (III) when the ligand is an amine, an amide, an N-heterocyclic carbene, cyclopentadienyl and its derivatives, nitriles, pyridine and its derivatives, bipyridines, preferably 1 to 6 molar equivalents, more preferably 1 to 4 molar equivalents.

12. The method according to claim 1, wherein R₁, R₂ and R₃ are identical and represent a hydrogen atom.

13. The method according to claim 1, wherein
R₁ represents a methyl radical, and R₂ and R₃ are identical and represent a hydrogen atom; or
R₃ represents a methyl radical, and R₁ and R₂ are identical and represent a hydrogen atom.

14. The method according to claim 1, wherein the catalyst is CO₂(CO)₈, NaCo(CO)₄, or [(C₆H₅)₃P)₂N]Co(CO)₄.

15. The method according to claim 1, wherein the metal catalyst is Pd(CF₃COO)₂.

16. The method according to claim 1, wherein the following occurs
with carbon monoxide or a mixture of carbon monoxide and hydrogen (CO/H₂);
in one or a mixture of solvents selected from acetonitrile, propionitrile and benzonitrile; and
in the absence of ligand.

17. The method according to claim 1, wherein it takes place in one or a mixture of at least two solvents chosen from aromatic hydrocarbons chosen from benzene and toluene;
with a mixture of carbon monoxide and hydrogen (CO/H₂); and
in the presence of phosphine or polyphosphine ligands.

18. (canceled)
